(19) United States
(12) Patent Application Publication
(10) Pub. No.: US 2012/0190989 A1
(43) Pub. Date: Jul. 26, 2012

(54) DISTRIBUTED EXTERNAL AND INTERNAL WIRELESS SENSOR SYSTEMS FOR CHARACTERIZATION OF SURFACE AND SUBSURFACE BIOMEDICAL STRUCTURE AND CONDITION

(75) Inventors: William J. Kaiser, Los Angeles, CA (US); Majid Sarrafzadeh, Anaheim Hills, CA (US); Denise Aberle, Los Angeles, CA (US); Maxim Batalin, San Diego, CA (US); Alireza Mehrnia, Los Angeles, CA (US); Ani Nahapetian, Glendale, CA (US); James Sayre, Agoura Hills, CA (US)

(73) Assignee: THE REGENTS OF THE UNIVERSITY OF CALIFORNIA, Oakland, CA (US)

(21) Appl. No.: 13/358,703

(22) Filed: Jan. 26, 2012

Related U.S. Application Data
(63) Continuation of application No. PCT/US2010/045784, filed on Aug. 17, 2010.
(60) Provisional application No. 61/234,494, filed on Aug. 17, 2009.
(60) Provisional application No. 61/234,506, filed on Aug. 17, 2009.
(60) Provisional application No. 61/234,524, filed on Aug. 17, 2009.

(51) Int. Cl. A61B 5/00; A61B 5/05; A61B 5/03; A61B 6/00; A61B 5/01

(52) U.S. Cl. 600/476; 600/300; 600/549; 600/561; 600/547

(57) ABSTRACT

Systems and methods are disclosed that use wireless coupling of energy for operation of both external and internal devices, including external sensor arrays and implantable devices. The signals conveyed may be electronic, optical, acoustic, biomechanical, and others to provide in situ sensing and monitoring of internal anatomies and implants using a wireless, biocompatible electromagnetic powered sensor systems.

### CROSS-REFERENCE TO RELATED APPLICATIONS

This application a 35 U.S.C. §111(a) continuation of PCT international application number PCT/US2010/045784 filed on Aug. 17, 2010, which is a nonprovisional of U.S. provisional patent application Ser. No. 61/234,494 filed on Aug. 17, 2009, and a nonprovisional of U.S. provisional patent application Ser. No. 61/234,506 filed on Aug. 17, 2009, and a nonprovisional of U.S. provisional patent application Ser. No. 61/234,524 filed on Aug. 17, 2009, each of which is incorporated herein by reference in its entirety. Priority is claimed to each of the foregoing applications.

The above-referenced PCT international application was published as PCT International Publication No. WO 2011/022418 published on Feb. 24, 2011 and republished on May 5, 2011, each of which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to sensing systems, and more particularly to wireless sensing systems for chronic condition treatment and monitoring.

2. Description of Related Art

Characterization of tissue and organ structures is of increasing importance to diagnosing and treating medical conditions. For example, bioelectrical impedance characterization of tissue and organ structures has demonstrated a remarkable range of capabilities from characterizing tissue wound characteristics through detection of sub-epidermal moisture to revealing gastric function.

Another treatment area where diagnostic characterization is of increasing importance is with orthopedic and dental implants. For example, total hip arthroplasty causes biomechanical changes in the normal femur, including a redistribution and concentration of stress. These mechanical alterations in the femur cause local remodeling and resorption that affect the geometry and mechanical properties of the bone. Using such implants in the long run will cause considerable pressure/friction/strain on the structure/joint and hence increased risk of wear or fracture or problematic structural variations. Findings now suggest that a significant number exhibit wear that causes serious problems, including particulate matter developed by wear which produces toxic reactions, which can have serious effects on the health of the patients. Implant failures include instability and dislocation, mechanical loosening, wear and corrosion and infection. As a result, over 50,000 replacements, i.e. revision, operations for hip implants are done annually, with an average cost of over 50,000 USD, totally in an annual cost of 2.5B USD for revision operations alone.

Patients, who are increasingly younger, are less compliant than desirable due to the fact that they can lose pain sensation in their affected joint. Additionally, the improvements in the joint surgeries have resulted in patients feeling better about their ability to use and hence put strain on those joints. Hence, compliance is a challenging issue. Additionally, there is a lack of information about the multiple decade long use of these prosthesis, as in the past patients who underwent this surgery only lived very short periods of time with them, as they were more common in the elderly.

One cause for problems is misalignment which is the result of improper surgery. This misalignment can results in a much greater amount of grating and even improper interaction with the bone. Toxic release occurs when metal to metal or metal-to-plastic grating or scraping causes the aluminum oxide ceramic underneath to be exposed and leads to aluminum debris release inside the body. This impact malfunction can lead to poisoning because of the materials used.

Another area of interest is chronic obstructive pulmonary disease (COPD), which is a progressive and debilitating disease affecting between 10 and 24 million adults in the United States alone, and is expected to become the third most common cause of death worldwide within the next decade [1,2]. One treatment technique, Bronchoscopic lung volume reduction (BLVR), involves placing a device bronchoscopically to obstruct airways subtending the most hyperinflated, emphysematous lung. The rationale is that endobronchial obstruction may promote collapse, improvements in the pressure relationships between lung and chest wall, or favorably alter lung recoil of the remaining lung to promote expiratory airflow. Different BLVR systems are currently in clinical trials, each with different mechanisms of action. Endobronchial one-way valve systems, which are placed in the proximal (lobar, segmental) airways, are designed to allow expiratory egress of air while preventing air from entering the target area during inspiration. The airway bypass system involves creating a shunt between a central airway and a target region of damaged, hyperinflated lung. A paclitaxel-eluting stent is placed in the fenestration to expand and maintain the new passage between the airway and adjacent lung tissue. The fenestration facilitates lung emptying, reducing FRC without altering lung recoil per se. Finally, biological sealant/remodeling systems act at the alveolar level to produce permanent damage in tissue [14]. A substance is introduced bronchoscopically and polymerizes distally at the target site to produce collapse and remodeling of lung over several weeks.

The typical patient undergoing Bronchoscopic lung volume reduction (BLVR) must be followed closely with routine surveillance visits to document changes in pulmonary function and to monitor for complications. These surveillance visits may not reflect the changes in lung function that are occurring in real time, both at rest and with exertion.

Accordingly, an object of the present invention is to provide improved sensing and detection systems for monitoring various tissues and anatomy within the body. Another object is an improved monitoring sensor system to identify and prevent failure in various implants. Another object is an implantable wireless sensing device to provide on-demand feedback on the status of COPD devices absent a visit to the clinic. Moreover, they can be used to assess functional derangements occurring in the context of altered symptoms, and to better marry physiologic information with symptoms in a way that cannot otherwise be captured. The classical outcomes measures used to monitor patients with endobronchial devices are measures of airflow, lung volumes and exercise testing, all of which require specialized equipment. At least some of these objectives will be met in the following description.

### BRIEF SUMMARY OF THE INVENTION

Systems and methods are disclosed utilizing wireless coupling of energy for operation and include a diverse range of architectures from wearable fabric (“smart patches”) to implantable devices. Signals conveyed by these devices include: electronic, with a broad spectrum of signals for tissue, organ, orthopedic device, and skeletal structure characterization, optical, with a broad spectrum of wavelengths as well as time and frequency domain resolution, angular resolution, and hybrid system that combine optical with signals from multiple domains; acoustic, including a broad spectrum of wavelengths and probe characteristics and may include evaluation methods for interrogating implant-bone and tissue interfaces, or methods that apply acoustic signal receivers to detect the acoustic signals that are signatures of wear conditions; biomechanical, where pressure and displacement are applied to tissue or joints to enable a non-invasive characterization of tissue characteristic, joint characteristics, vascularity, and others. These also may be applied in a hybrid manner where tissue compression is combined with optical probes, for example, to determine characteristics of blood perfusion.

An aspect of this invention is the in situ sensing and monitoring of skin or wound or ulcer status using a wireless, biocompatible RF powered sensor system referred to as smart patch, smart band-aid or smart cast. This invention enables the realization of smart preventive measures by enabling early detection of infection or inflammatory pressure which would otherwise have not been detected for an extended period or may have required removal of a bandage for inspection with increased risk of infection as a result of the inspection process and wound or injury exposure.

In one beneficial embodiment, the inventive smart patch incorporates wireless sensing components to monitor and measure alterations in wound or skin characteristics including, but not limited to, moisture, temperature, pressure, surface electrical capacitance and/or bioelectric impedance.

Another aspect is an interrogatable external sensor system for acquiring one or more biological characteristics of a surface or internal tissue region of a body of a patient, comprising: a sensor array and an interrogator configured to transmit energy in the form of an electromagnetic waveform. The sensor array comprises: a substrate configured to be positioned external to and proximal to the patient's body; a plurality of sensor elements coupled to the substrate; a processor coupled to the substrate and connected to the plurality of sensor elements, wherein the processor is configured to communicate with at least one of the sensors elements in the array. Further, the sensor elements are configured to emit or receive a physiological signal through the internal tissue region or at a surface tissue region, wherein the physiological signal comprises at least one physiological characteristic of the surface or internal tissue region; and an antenna coupled to the array. The antenna is responsive to electromagnetic energy transmitted from the interrogator; wherein the electromagnetic energy powers the array with sufficient energy to power the emission or reception of the physiological signal through at least one of the sensor elements.

Another aspect is method for acquiring one or more biological characteristics of a surface or internal tissue region of a patient. The method includes the steps of positioning a sensor array external to and adjacent to a region of the patient's skin, wherein the array comprises a plurality of sensor elements connected to a processor. The method further includes the step of positioning an interrogator in proximity to the array, wherein the interrogator is configured to transmit energy in the form of an electromagnetic waveform. Further steps include, transmitting an electromagnetic signal from the interrogator, receiving the electromagnetic signal via an antenna coupled to the array, inductively powering the array via the electromagnetic signal, and instructing the array via the electromagnetic signal to emit or receive a physiological signal through the internal tissue region or at a surface tissue region, wherein the physiological signal comprises at least one physiological characteristic of the surface or internal tissue region.

Another aspect is a transdermal sensor system for acquiring one or more biological characteristics of an internal tissue region of a patient, comprising: an interrogator configured to transmit energy in the form of an electromagnetic waveform; an external sensor array; an implant disposed at or near the internal tissue region; wherein the implant comprises at least one internal sensor element configured to exchange a transmissive physiological signal through the internal tissue region with the external sensor array; wherein the physiological signal comprises at least one physiological characteristic of the internal tissue region; wherein the implant comprises an internal antenna responsive to electromagnetic energy transmitted from the interrogator; and wherein the electromagnetic energy powers the implant with sufficient energy to power the exchange of the physiological signal through the at least one internal sensor element.

Another aspect is a method for acquiring one or more biological characteristics of an internal tissue region of a patient. The method includes the steps of positioning a sensor array external to and adjacent to a region of the patient's skin, delivering an implant to a location at or near the internal tissue region, positioning an interrogator in proximity to said array, wherein the interrogator is configured to transmit energy in the form of an electromagnetic waveform and the implant comprises an internal antenna responsive to electromagnetic energy transmitted from the interrogator. Further steps include transmitting an electromagnetic signal from the interrogator, receiving the electromagnetic signal via the internal antenna, inductively powering the implant via the electromagnetic signal, and instructing the implant via the electromagnetic signal to exchange a physiological signal with the external array through at least a portion of the internal tissue region, wherein the physiological signal comprises at least one physiological characteristic of the internal tissue region.

A further aspect is an interrogatable sensor system for acquiring one or more biological characteristics of an internal tissue region of a patient, comprising: an interrogator configured to be positioned at a location external to the body of the patient and transmit energy in the form of an electromagnetic waveform; a first implant configured to be disposed at or near the internal tissue region; wherein the first implant comprises a sensor element configured to receive a physiological signal through at least a portion of the internal tissue region; wherein the physiological signal emanating within the body of the patient and comprising at least one physiological characteristic of the internal tissue region; wherein the first implant comprises an antenna responsive to electromagnetic energy transmitted from the interrogator; and wherein the electromagnetic energy powers the implant with sufficient energy to power the receipt of the physiological signal through the sensor element.

Yet another aspect is a method for acquiring one or more biological characteristics of an internal tissue region of a patient, comprising the steps of positioning an interrogator at a location external to the body of the patient, wherein the interrogator is configured to transmit energy in the form of an electromagnetic waveform, and delivering a first implant to a location at or near the internal tissue region, wherein the first implant comprises a sensor element configured to receive a physiological signal through at least a portion of the internal tissue region and an antenna responsive to electromagnetic energy transmitted from the interrogator. The method further includes the steps of transmitting an electromagnetic signal from the interrogator, receiving the electromagnetic signal via the antenna, inductively powering the first implant via the electromagnetic signal, and instructing the implant via the electromagnetic receive a physiological signal emanating within the body of the patient and comprising at least one physiological characteristic of the internal tissue region, wherein the electromagnetic energy powers the implant with sufficient energy to power the receipt of the physiological signal through the sensor element

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 illustrates a perspective view of the components of an external sensor system “extrasensor” and interrogator in accordance with the present invention.

FIG. 2 is a schematic diagram of the external sensor system of FIG. 1 operated in a reflective mode.

FIG. 3 is a schematic diagram of the external sensor system of FIG. 1 operated in a passive mode.

FIG. 4 is a schematic diagram of the external sensor system of FIG. 1 operated in a transmissive mode with another external sensor patch or external device

FIG. 5 illustrates a freeform external sensor array in accordance with the present invention.

FIG. 6 illustrates a radial external sensor array in accordance with the present invention.

FIG. 7 illustrates a perspective view of the components of a transdermal sensing system “intrasensor” with an external sensor directing transmissions into the body in accordance with the present invention.

FIG. 8 illustrates a perspective view of the transdermal sensing system of FIG. 7 with an external sensor receiving transmissions from intrasensor implants with the body.

FIGS. 9 and 10 illustrate embodiments of a transdermal sensing system with intrasensor implants positioned in various locations within a prosthetic hip implant in accordance with the present invention.

FIG. 11 illustrates a schematic diagram of the components of a transdermal sensing system in accordance with the present invention.

FIG. 12 is a schematic perspective view of the intersensor system “intersensor” with implanted intersensor devices operating in a transmissive mode in accordance with the present invention.

FIG. 13 is a schematic diagram of the components of intersensor system in accordance with the present invention.

FIG. 14 is a perspective schematic view of an intersensor stent in accordance with the present invention.

FIG. 15 a schematic diagram of the components of intersensor stent of FIG. 14 with interrogator.

FIG. 16 illustrates an intersensor implant installed within a passageway of the lung in accordance with the present invention.

### DETAILED DESCRIPTION OF THE INVENTION

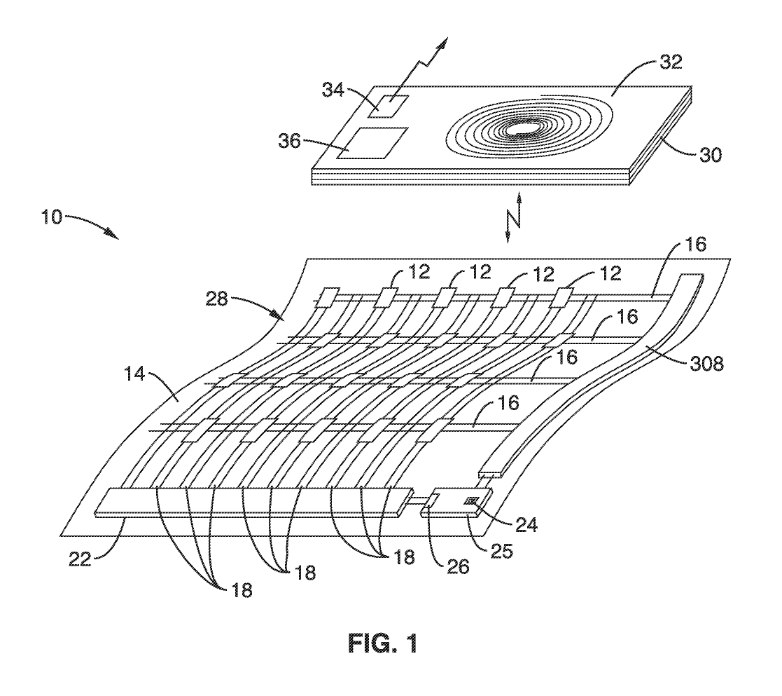

Referring more specifically to the drawings, for illustrative purposes the present invention is embodied in the apparatus generally shown in FIG. 1 through FIG. 16. It will be appreciated that the apparatus may vary as to configuration and as to details of the parts, and that the method may vary as to the specific steps and sequence, without departing from the basic concepts as disclosed herein.

1. ExtraSensor System

FIG. 1 illustrates the “ExtraSensor” or external sensing system 10 in accordance with the present invention. For purposes of this description, “Extrasensor” devices are defined as externally applied, compact devices that are externally powered via an interrogator.

External sensing system 10 comprises an array 28 of nodes 12 positioned at the locations of intersections of row 16 and column 18 transmission lines.

The array 28 is preferably positioned on a substrate 14 that supports the array and other analog and digital components. The substrate 14 preferably comprises a flexible and biocompatible material such as laminated Kapton (polymide) chip-on-flex which conforms to the applied surface. This enables various different modes of use including, but not limited to, a band-aid, cast, patch, tissue, etc. The flexible substrate 14 also permits the external patch 10 to be applied directly in single or multiple units, or incorporated into adhesive patches, garment systems, shoe systems, and other wearable items in methods familiar to those skilled in the art.

Each node 12 comprises a sensor element or emitter element for respectively receiving or transmitting a signal. The nodes 12 may alternate between sensor elements and emitter elements, or comprise both an emitter and sensor at each node. Alternatively, the array 28 may be a population of nodes 12 with sensor and emitter elements and with a node spatial density adapted to best serve application measurement requirements. In one embodiment, each node 12 may comprise a switching element (that may include, for example, a field effect transistor switch or the like) that is coupled to a respective emitter element or sensor element. Each node 12 is coupled, via row and column transmission lines 16 and 18 and row and column ribbons 22, 20, to an internal processor 26. The internal processor 26 drives operation for reception or transmission of signals through the emitter or sensor in each node 12, wherein the array 28 may be accessed to read data in a programmable and multiplexed manner.

Alternatively, each node 12 may comprise a complete digital and analog processing system may be included that comprises a signal generator and a signal receiver. The signal generator produces a signal applied to the emitter nodes 12 at the row and column intersections to produce a signal that propagates into adjacent tissue. Also, the signal receiver acquires signals via dedicated sensor nodes as well.

The above embodiments enable the measurement of displacement current at the sensing element nodes 12 (when isolated from tissue by a spacing or by an insulator layer), and also current associated with direct contact with tissue as determined by application needs.

The external sensor 10 is configured to receive operating energy by direct, wireless coupling to an electromagnetic signal source and not requiring a wireline connection to a signal source. In a preferred embodiment, an interrogator 30 is used to transmit energy to the sensor pad 10 via antenna 24 on battery-less integrated circuit die 25. A tissue scanning operation may be initiated by the interrogator 30, which excites the on-surface coil/antenna 24 embedded in the integrated circuit die 25 and provides the needed energy burst to support the scanning/reading operation.

In a preferred embodiment, the array 28 is powered by radio frequency (RF) coil antenna 32 in the interrogator, which directs radio frequency (RF) energy to embedded sensor array 28 via a receiving antenna 24. The supplied transmission powers the on-board integrated circuit 25 and sensor array 28, without the need of a battery. For example, upon a scanning operation initiated by the interrogator 30, the on-surface coil 24 embedded in the external patch 10 is excited, and provides the needed energy burst to support the scanning/reading or other control operations. Interrogator 30 may be a handheld device, or can be worn as a belt or integrated with a smart phone via USB, Bluetooth or other connection.

Upon reception of a trigger from the interrogator 30, the integrated circuit processor 26 addresses the sensors/emitter nodes 12 and reads their measurements of surface/wound/tissue characteristics. Such characteristics may include, but are not limited to, temperature, moisture, pressure, bioelectric impedance, and electrical capacitance, spectroscopic or optical features, described in further detail below.

In a preferred embodiment, the array 28 has the flexibility of embedding various sensor/emitter types at nodes 12 to enable simultaneous reading of any combination of the aforementioned characteristics to enable fusion on captured information for better decision making and wound management.

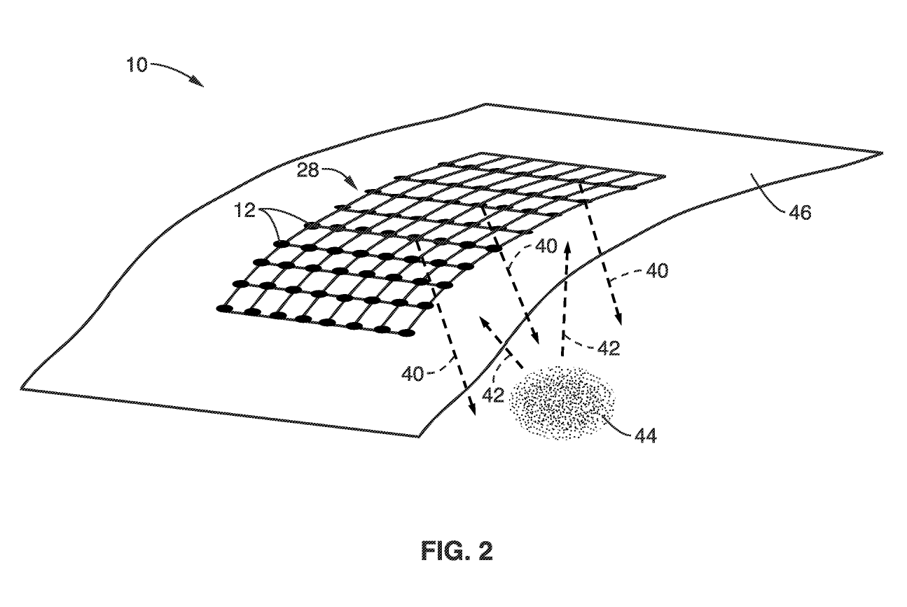

FIGS. 2 through 4 illustrate various diagnostic/treatment modalities for an external patch 10 in accordance with the present invention. As shown in FIG. 2, the patch 10 may be positioned adjacent or in proximity to a patient's skin 46 or other body part (e.g. eye, tooth etc.), such that the array 28 may operate in a reflective mode generally parallel to the skin surface 46. One or more nodes 12 may be directed to emit a signal 40 into the body of the patient in the direction of an anatomical region of interest (e.g. body part, implant, tumor etc.). Reflected rays 42 are then received from sensor nodes 12 that provide useful data about the region of interest 44. For surface detection, it is appreciate that the emitted signal 40 does not penetrate, or substantially penetrate the skin, such that the reflected rays 42 are merely reflected from the skin surface.

It is understood that the beam patterns or rays 40, 42, 46, 48, 74 and 78 shown in FIGS. 2-4 and 7-8 are intended to indicate the direction of the probing signal, and not the actual beam pattern, nor restrict the special distribution beam pattern (e.g. beam swath may be conical). For purposes of illustration, only the array pattern of the external sensing device 10 is shown.

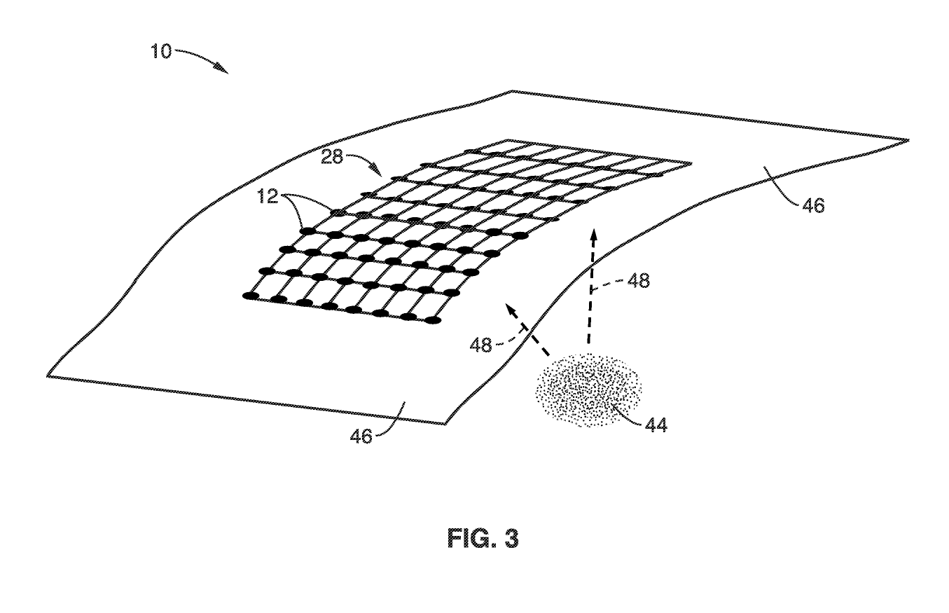

Referring to FIG. 3, the external patch 10 may be operated in a passive mode, wherein rays emanating 48 from a region of interest 44 may be sensed by one or more sensing nodes 12 of the array. For example, the external patch 10 may operate as a passive electronic spectroscope to retrieve and measure and monitor signals generated by a subject's internal organs in a passive fashion without application of an external signal. This may be combined with the bioelectrical impedance, optical, and acoustic systems, or may operate independently.

In one embodiment, the passive external sensor 10 may be applied to detect signals arising from a cardiac sinoatrial node pacemaker, signals arising from cerebral function as applied in electroencephalography, and those appearing from skeletal muscle function as applied in electromyography. Other applications may comprise general electrocardiography, electrooculography, electroretinography, and audiology.

In a preferred embodiment, the external patch 10 is configured for bioelectrical impedance characterization of tissue and organ structures, wherein the node elements 12 comprise electrode sensors and emitters, and an electric current is delivered to the nodes 12 of the matrix array 28 via electrically conductive row and column connector wires 16 and 18. Electrode nodes 12 may be directly coupled to tissue and many include the materials familiar to those skilled in the art for enhancing either conductive or capacitive coupling.

The biometric impedance probe allows for direct measurement of bioelectrical impedance over a wide frequency range. Exemplary applications may include measurement of sub epidermal moisture or gastric function. A plurality of external patches may be applied to permit measurement of impedance coupling, for example, of the entire abdomen of a subject to monitor of gastric function.

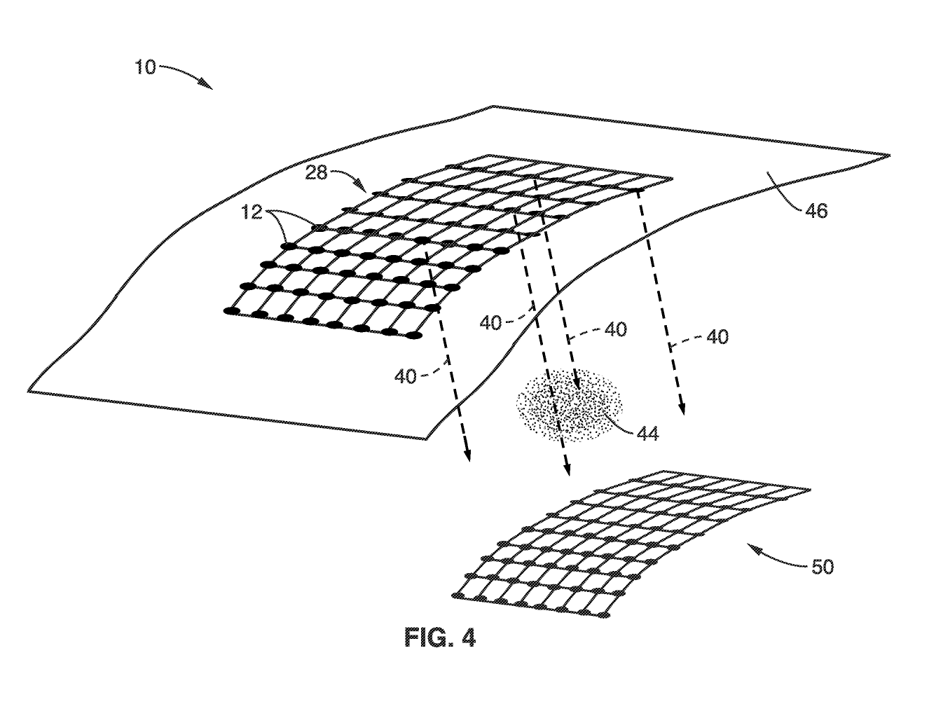

As shown in FIG. 4, an additional external sensor patch 50 (or other external source) may be used in transmissive operation to characterize transmitted signals 40 through a tissue region of interest 44.

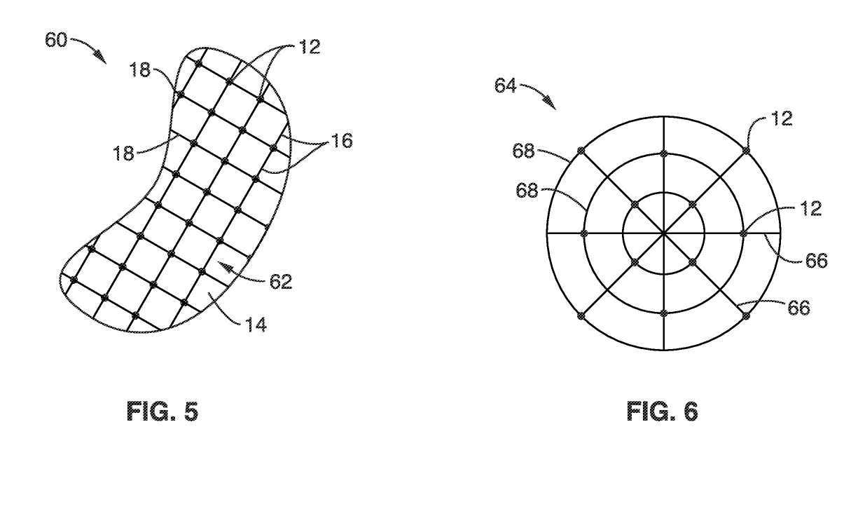

While the external sensor patch 10 is depicted as a rectangular array 28 in FIGS. 1-4, and 7-8, it is appreciated that the array 28 may comprise any number of shapes. For example, FIG. 5 shows a free-form array 60 positioned on a substrate 14 that is shaped to conform to a particular anatomical feature. The array 60 may comprises row 16 and column 18 transmission lines to the individual nodes. Alternatively, The array may be radial, as shown in FIG. 6, wherein array 64 comprises nodes 12 at intersections of radial spokes 66 and concentric axial circles 68.

The external sensor system 10 also includes analytical software modules (e.g. stored in memory in circuitry 36 of the interrogator 30), with signal processing to characterize frequency dependent, and complex (as in both real and imaginary part) impedance characteristics of the subject tissue 44 or body structure under evaluation. The interrogator 30 may also include a second antenna 34 the communicates wirelessly (e.g. via WIFI, Bluetooth, etc.) to couple to external network devices supplying resources that may provide additional signal processing, or provide reception of data processed by the external sensing system 10. This also includes control systems that determine signal waveforms including frequency, amplitude, and other signal modulation characteristics.

The external bioelectrical impedance system 10 may also incorporate amplitude, frequency and time domain diversity in measurements. For example, those skilled in the art will be aware that the amplitude, frequency, and time sequence of signals may be applied to characterize tissue. For example, by varying signal frequency, the frequency-dependent dielectric response of tissue will enable control of depth resolution for measurements. Further, by monitoring signal phase, then both real and imaginary components of dielectric response are revealed using methods again familiar to those skilled in the art of impedance spectroscopy.

The external sensing system 10 may also operate in combination with the delivery and application of therapeutic agents or other materials to a tissue treatment site 44 of interest, where such agents may comprise biochemical compounds or pharmaceuticals. These agents can be delivered externally, by injection and specific locations, or ingested. In each case, the response of tissue characteristics to the application may be helpful in detecting further tissue properties.

The external sensing system 10 may also operate in combination with applied mechanical pressure. For example, the application of pressure to tissue results in a reduction of blood perfusion in the region of applied pressure to a degree and with a time response that may reveal the state of tissue. The external bioelectrical impedance probe 10 is configured to measure the response of this tissue region through a method that includes application of pressure to the external patch 10, which may optionally include integral pressure sensors (not shown). The bioelectrical impedance signal may be modulated by the change in subsurface fluid density, which reflects change in perfusion or change in tissue edema conditions.

The external sensor system 10 may also include protective sheath materials or covering materials (not shown) that are permanent or temporarily applied, or may be disposable in nature. This permits the external sensor system 10 to be used in applications where the array elements 12 are isolated from the tissue surface 46 and equipped with a disposable protective sheath that is replaced between usages. The choice of materials for this isolation may include elastomers, other materials known in the art.

The external sensor system 10 may also include pressure sensors (e.g. thin film polymer devices) or conductive or capacitively coupled electrodes or optical elements, detect alarming pressures in scenarios similar to pressure ulcer patients and monitor local blood circulation status. The pressure sensors may also be used to verify the placement of the external sensor system 10 at the target site of measurement. These elements may be also used to show that both placement and orientation of the external patch 10 is verified according to a prescribed application by using methods for position verification readily familiar to those skilled in the art.

The external sensor 10 may also be equipped with external markings (e.g. a radio-opaque marker at the corners or outline of the flexible substrate 14) that permit verification of application positioning using external imaging systems.

The external patch 10 may also include an indicator (e.g. light emitting diode (LED), not shown) on its visible surface which may illuminate upon detection of a target event by the corresponding sensors on the other side of the patch.

In an alternative embodiment, the external sensor 10 may also contain super capacitor or battery element to enable extended operation during intervals of time that occur between events when RF energy is delivered providing energy for charging of capacitor or battery elements as will be obvious to those skilled in the art

The External sensor system 10 of the present invention promotes better management of each individual patient, resulting in a more timely and efficient practice in hospitals and even nursing homes. This is applicable to patients with chronic wounds, diabetic foot ulcers, pressure ulcers, post-operative wounds, accidental injuries or bone fracture. In addition, alterations in signal content may be integrated with the activity level of the patient and standardized assessments of symptoms.

Retrieved data from patients may be stored and maintained in a signal database, such that pattern classification, search, and pattern matching algorithms may be used to better map symptoms with alterations in wound or skin characteristics.

It is appreciated that the external sensing system 10 of the present invention may be used for diagnosing and treatment of specific ulcer (e.g. diabetic foot ulcer, pressure ulcer, or the like) or chronic wound conditions (e.g. stage III and stage IV pressure ulcer cases, which are a major cause of mortality in the bedridden senior patients), post-operative wounds, accidental injuries or broken limbs, in addition to broad application in all forms of arthritis and even skin diseases.

In one embodiment, the array 28 of the external sensing system 10 may be configured to act as thermal sensor to sense and read skin, tissue or wound thermal data, as wound status is often correlated with wound's thermal data. Furthermore, external sensing system 10 may detect and moisture status of skin or tissue to monitor redness, swelling or arthritis and prevent infection.

In another preferred embodiment, the array 28 of the external sensing system 10 may be configured to operate as an optical spectroscope. This may be combined with the previously described bioelectrical impedance system, or operate independently. In such an embodiment, nodes 12 comprise optical sensors and emitters at the site of each row 16 and column 18 of the matrix array 28, or at selected sites.

Optical sensors may include photodiodes, including those with specified narrow band or broad band spectral response and those optimized for high time resolution for detection of temporally short optical pulses and signal systems requiring high time resolution. Emitters may include light emitting diodes (LED's) operating over a range of wavelengths and those that may be equipped with narrow band optical filters. Further, emitters may include semiconductor laser systems.

Transmission lines 16 and 18 may comprise fiber optic lines or means for delivery of optical signals at the node 12 locations. Fiber optic means may also be applied to acquire optical signals that may then be supplied to external spectroscopic resolving equipment (not shown). The external sensor assembly 10 may also be configured to operate with separate optical sources (not shown), wherein the sensor assembly array 28 is predominantly equipped with optical detectors at nodes 12 to receive optical transmissions from the external source. Accordingly, the sensor assembly array 28 may be predominantly equipped with optical transmitters at nodes 12 to transmit optical transmissions to optical detectors on an external source (see e.g. transmission rays 44 in FIG. 4).

External interrogation via interrogator 30 may also be realized through directing EM energy in the optical (infrared, visible, ultra-violet) frequency range, to both power and communicate with the on-board sensor array integrated circuit die 25. In such configuration, the antenna 24 may comprise a photodiode receptor or the like.

In one embodiment, spectroscopy means may also be applied to both detector and emitter nodes 12. This includes the use of multiple devices and filters to resolve the propagation of optical signals through tissue 44. The arrangement of sensors and emitters also includes a diversity of emitter and receiver pairs at nodes 12 with varying angular emittance to enable detection of phenomena at varying depth and location.

Detection and analysis methods known in the art and based on infrared signal absorption may also be used to resolve the presence of subsurface oxyhemoglobin and deoxyhemoglobin to, for example, detect subsurface blood perfusion state. The emitter and detector deployment pattern 28 may be adapted to enable detection of specific tissue regions.

Optical signals may also be applied to induce fluorescence in tissue or in materials applied to tissue, injected, or delivered as a pharmaceutical to a subject. These materials may include biochemical compounds. Nonlinear optical phenomena (for example that of Raman spectroscopy) may be used to further characterize of tissue or detection of specific materials.

Referring back to FIG. 2, the optical spectroscopy of external sensor 10 may be applied in a reflective mode (where sensors and emitter nodes 12 are dispersed within the same array 28 to generate signals 40 that are reflected as light beams 42).

Referring back to FIG. 4, the optical spectroscopy of external sensor 10 may also be applied in transmissive (e.g., a plurality of external sensors 10 are applied to enable spectroscopic interrogation of tissue by optical transmission beams 40).

In another preferred embodiment, the external sensor system 10 may be configured as a passive or active acoustical spectroscope with use of acoustic sensors and emitters at nodes 12 of the matrix array 28.

In a passive mode of operation, the external sensor system 10 equipped with acoustic sensors at one or more of the nodes 12 that are configured to detect acoustic signals or mechanical vibration signals that arrive at the site of the sensor array 28 after passing through tissue (e.g. beams 48 emanating from an anatomical target area 44, as shown in FIG. 3). The external sensor system 10 may be attached as part of a smart patch integrated with garments, shoes or other wearable systems. Alternatively, the external sensor system 10 may be applied by direct application as a handheld instrument to tissue. Acoustic signal or vibration signal detection may operate over a frequency range spanning from very low frequency (e.g. 10 Hz or less) to high frequency ultrasound (greater than 100 MHz). Acoustic sensors may be applied directly to tissue and may also incorporate impedance matching layers separating the sensor array 28 from tissue surface 46.

A preferred embodiment of a passive acoustic external sensor 10 may be to detect the vibration signals and acoustic emission signals that are typical of mechanical wear associated with bearing surfaces (e.g. region 44 in FIG. 3). This permits the detection of wear indication associated with biomedical implant devices whether associated with joints (knee or hip) or dental implants. Condition based monitoring (CBM) principles, as available in the art, may be applied for such detection.

It is important to note that in this preferred embodiment, the external system 10 may be combined with mechanical manipulation or motion of limbs and joints to enable detection of conditions of joints, implants, or other structures revealed by the acoustic emission that occurs in the event of motion.

In one preferred embodiment, an active acoustic external sensor assembly 10 includes narrow band or broadband acoustic transducers operating at low or high frequency, and placed at specified nodes 12 along with acoustic sensor elements within the array 28. In this preferred embodiment, the external sensor assembly 10 may then be applied to external tissue 46 create acoustic signals 40 that propagate into tissue via the acoustic emitters (see FIG. 2). The reflected acoustic signals 42 are then detected as signals reflected from subsurface tissue and subsurface physiological structure 44 (for example that of tissue, skeletal bone, subsurface organs, or implanted devices that may include orthopedic devices).

In a further configuration, more than one external sensor system 10 may be applied to permit characterization by transmission of acoustic signals 40 (as shown in FIG. 4). This embodiment enables characterization of tissue, interrogation of skeletal bone condition associated with (for example) bone fracture healing, and interrogation of implant status. Monitoring of cardiac, arterial, pulmonary, and gastric systems may also be performed.

2. IntraSensor System

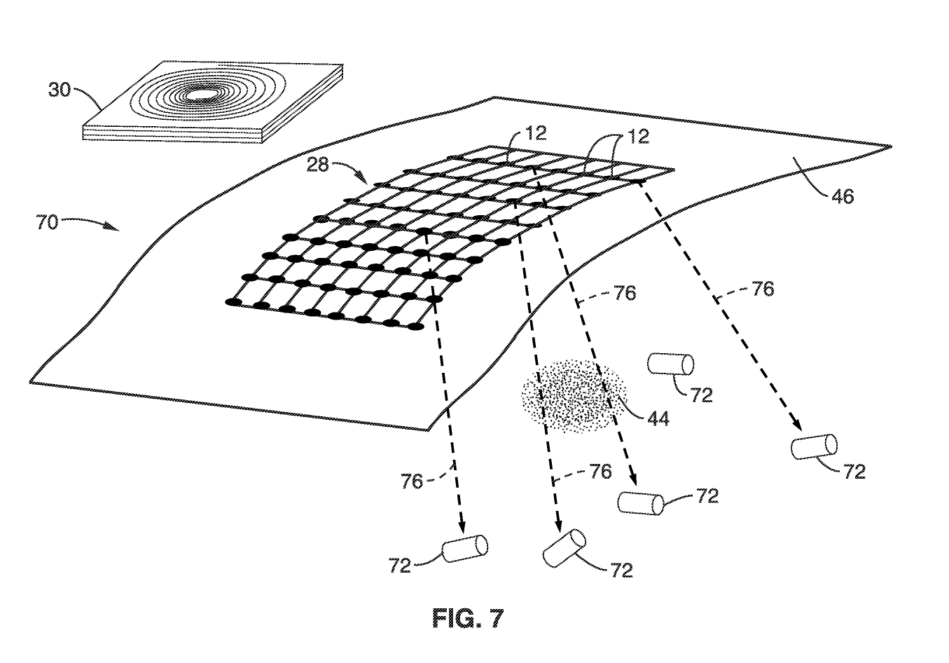

FIGS. 7 through 11 illustrate the “Intrasensor” system of the present invention. For purposes of this description, an “IntraSensor” is defined as a hybrid sensor system that incorporates an external element applied externally to tissue that sends and or receives physiological data signals via a transdermal communication between one or more implanted elements below the tissue surface and/or integrated directly with orthopedic implants associated with (for example) skeletal joints or dental systems. The “IntraSensor” implants are primarily composed of systems that derive operating energy from the receipt of externally applied electromagnetic signals (e.g. radio frequency (RF) energy).

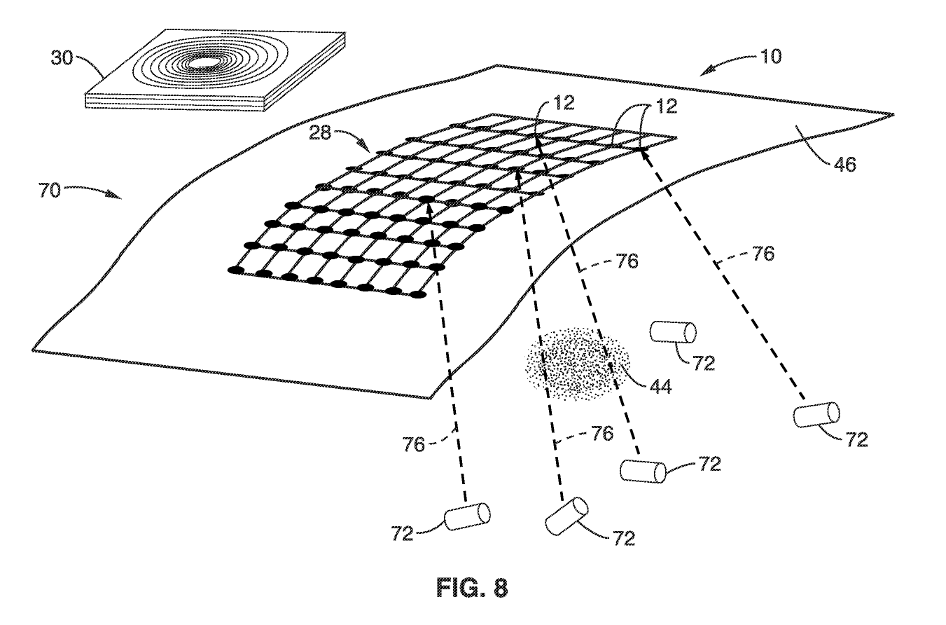

Referring now to FIG. 7, a transdermal sensor system 70 includes one or more external sensor assemblies (for example, but not limited to, the Extrasensor system 10 shown in FIGS. 1-6) and one or more implantable sensor emitter devices 72. FIGS. 7 and 8 show an external sensor assembly 10 having an array 28 of sensing/emitting nodes 12 that lie adjacent skin surface 46. In FIG. 7, the array 28 is emitting one or more signals from the nodes 12 through the skin toward an array of individual sensor implants 72 configured to receive the transmitted signal. In FIG. 8, the array 28 is receiving one or more signals 74 from the nodes 12 through the skin from an array of individual sensor implants 72 configured for signal emission.

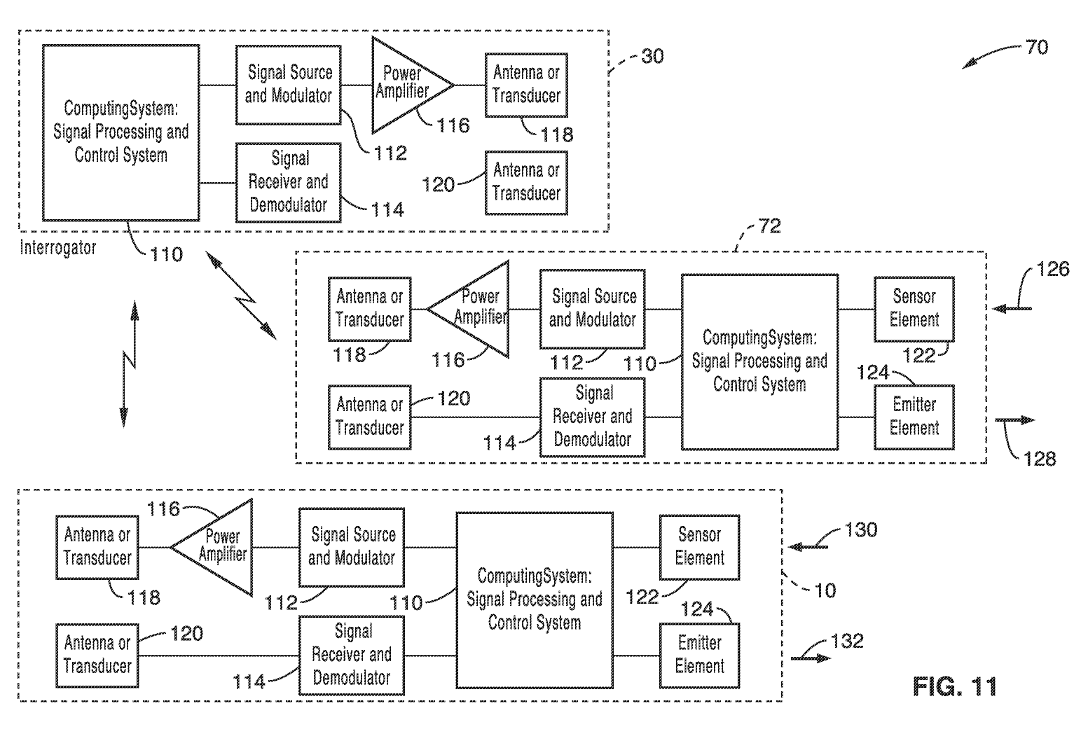

FIG. 11 illustrates a schematic diagram of the primary components of a transdermal sensor system 70 in accordance with the present invention. Transdermal sensor system 70 includes an interrogator 30 that is configured to communicate with and provide power to an external sensor system 10 and one or more intrasensor implants 72. It is appreciated that the interrogator 30 may be integrated with or operate in a separately applied package from the external sensor system 10. The interrogator 30 provides the source energy (e.g. radio frequency (RF) electromagnetic signals) and communication for operation of the external sensor system 10 and one or more intrasensor implants 72. Even in the event that the interrogator 30 is separately packaged, its operation can enable communication with the external sensor system 10 to permit time synchronized and time and event coordinated operation external sensor system 10 and intrasensor implants 72.

As shown in FIG. 11, the interrogator 30 includes a processor 110 for commanding and controlling the operation of intrasensor implant 72 elements and external sensor system 10 elements according to a sequence of operations upon a set of programming instructions stored within memory on the interrogator 30 (e.g. via board 36 shown in the interrogator 30 of FIG. 1), or provided to the interrogator from an outside source. The processor 110 is also configured to receive, process, and store information from intrasensor implant 72 and external sensor system 10.

The interrogator 30 further includes a signal generator and modulator 112 to permit the transmission of data. A power amplifier 116 amplifies the modulated signal, which is then transmitted via antenna or transducer 118 for reception by the intrasensor implant 72 and/or external sensor system 10.

In a preferred embodiment, the signal generator and modulator 112 are configured to generate a radio frequency (RF) electromagnetic signals. In such configuration, the antenna 118 may comprise a coil antenna 32 (as shown in shown in interrogator 30 of FIG. 1), configured to generate the radio frequency signal.

The interrogator 30 further includes an antenna or transducer 120 to receive communication transmissions from either the external sensor system 10 and/or intrasensor implants 72. The antenna 120 is coupled to a signal receiver and demodulator 114 to demodulate the radio frequency signal so as to permit the reception and recovery of data for processor 110. In an alternative embodiment, it is possible that only one antenna (e.g. antenna 118) is used for both transmission and reception of signals.

Each intrasensor implant 72 comprises a processor 110 for commanding emitter element 124 and receiving data from sensor element 122 with regard to their sequence of operations to affect the desired physiological measurements within the target tissue. For example, the emitter element 124 may emit a signal 128 into and through an adjacent region of tissue. In reflective operation the emitted signal may be reflected back as signal 126 to be received by sensor element 122.

Alternatively, in a transmissive operation, the emitted signal 128 is received as incoming signal 130 by sensor element 122 of external sensor 10. It is also appreciated that the intrasensor implant 72 may only comprise one of either an emitter element 124 or sensor element 122 for one way transmissive communication with the external sensor 10.

The intrasensor implant 72 is capable of receiving data, information or commands from interrogator 30 via antenna or transducer 120. This data is received and demodulated at 114 to rectify the signal properly to derive potentials that may enable operation of microelectronic circuits.

The intrasensor implant 72 further includes a signal generator and modulator 112 to permit the transmission of data back to the interrogator 30. A power amplifier 116 amplifies the modulated signal, which is then transmitted via antenna or transducer 118 for reception by the interrogator 30.

The external sensing system 10 comprises a processor 110 for commanding emitter element 124 and receiving data from sensor element 122 with regard to their sequence of operations to affect the desired physiological measurements within the target tissue. For example, the emitter element 124 may emit a signal 132 into and through an adjacent region of tissue.

In reflective operation (assuming the external sensor system is the sole unit being used as shown in FIG. 2) the emitted signal 132 may be reflected back as signal 130 to be received by sensor element 122.

Alternatively, in a transmissive operation via transdermal system 70, the emitted signal 132 is received as incoming signal 126 by sensor element 122 of intrasensor implant 72. It is also appreciated that the external sensor 10 may only comprise one of either an emitter element 124 or sensor element 122 for one way transmissive communication with one or more of the intrasensor implants 72.

Although FIG. 11 only shows one emitter element 124 and sensor element 122 for external sensing system 10, it is appreciated that the external sensing system 10 may comprise a plurality of elements 122, 124 positioned on nodes 12 of the array 28 (and alternatively arrays 60 and 64) detailed in any of FIGS. 1-8.

The intrasensor implant 72 is capable of receiving data, information or commands from interrogator 30 via antenna or transducer 120. This data is received and demodulated at 114 to rectify the signal properly to derive potentials that may enable operation of microelectronic circuits.

The intrasensor implant 72 further includes a signal generator and modulator 112 to permit the transmission of data back to the interrogator 30. A power amplifier 116 amplifies the modulated signal, which is then transmitted via antenna or transducer 118 for reception by the interrogator 30.

In a preferred embodiment, the interrogator 30 shown in FIG. 11 comprises means to convey energy from the Interrogator device (located external to tissue) to subsurface intrasensor implants 72 and external sensor 10. This energy is preferably in the form of an electromagnetic signal (e.g. RF) similar to RFID technology. The intrasensor implant 72 and external sensor system 10 include a means (e.g. antenna 120) to recover energy from the received electromagnetic signal in order to provide the respective device with required energy for its operation. Such energy recovery may be based on methods for rectification of RF signals available in the art.

Further, the intrasensor implant 72 and external sensor system 10 comprise a means (e.g. antenna/transducer 118) to produce an electromagnetic signal comprising a data communication carrier signal that may be received by the interrogator 30 for the purposes of conveying information from the either the intrasensor implants 72 and external sensor 10 to the Interrogator. This information may include data describing the signals associated with sensor and emitter elements 122 and 124.

The data communication carrier signal described above preferably comprises an electromagnetic propagating wave as familiar to those skilled in the art of RFID technology. However, it is appreciated that the data communication carrier may be an optical, acoustic, or other signal that provides an adequately reliable data communication channel. This data communication carrier signal may also convey energy as required or operation of the intrasensor implant 72 and/or external sensor system 10. For example, where an electromagnetic propagating wave is replaced by optical, acoustic, or other signals, then appropriate transducers for respectively, optical (e.g. photodiode emitters and sensors) or acoustic (e.g. ultrasound emitters and sensors), or other signals will vary accordingly for respective receipt of signals and conveyance of necessary energy.

In one embodiment, the interrogator 30, intrasensor implant 72 and/or external sensor system 10 may only use a single antenna or transducer to combine the roles of signal transmission and reception. However, antennas or transducers may be selected to best optimize operation.

The interrogator 30 enables the communication of data from the interrogator computing system or processor 110 to the computing systems of the intrasensor implant 72 and/or external sensor system 10. This occurs via generation of data, modulation of this data onto a data communication carrier signal, introduction of a power amplification step, and finally the emission of this data from an antenna or appropriate transducer and its propagation to the intrasensor implant 72 and/or external sensor system 10. At the intrasensor implant 72 and/or external sensor system 10, this data communication carrier is received, demodulated and made available as data to the computing system that is part of the respective intrasensor implant 72 and/or external sensor system 10. Finally, the data transmitted between interrogator 30 and intrasensor implant 72 and/or external sensor system 10 may include sensor measurement data associated with physiological signals (including those associated with bioelectric impedance, optical spectroscopic, or acoustic spectroscopic). The data transmitted between interrogator 30 and intrasensor implant 72 and/or external sensor system 10 may also include program sequence instructions intended to be applied by the computing system of the respective interrogator 30 and intrasensor implant 72 and/or external sensor system 10 for control of both the function of emitter and sensor elements.

Finally, the intrasensor implant 72 and/or external sensor system 10 include emitter and sensor elements 122, 124 that generate and receive signals including those associated with bioelectric impedance, optical spectroscopic, or acoustic spectroscopic. These signals propagate between intrasensor implant 72 and/or external sensor system 10 elements, or between the intrasensor implant 72 and/or external sensor system 10.

In one preferred embodiment, multiple intrasensor implants 72 operate in sequence or simultaneously with data that may be combined via sensor fusion methods for inference of internal organ state.

The intrasensor implant 72 elements 122, 124 may contain two or more electrodes that are either insulated from or in contact with internal tissue. The intrasensor implant 72 elements 122, 124 in this embodiment may include a dedicated digital control system and wireless communication interface that enables control and coordination with external devices through a communication channel conveyed via the same radio frequency signal applied for energy transmission, or a separate channel. This communication channel in this embodiment may exploit means that are familiar to those skilled in the art of RFID technology.

The intrasensor implant 72 elements 122, 124 may generate an electronic signal that is coupled to tissue via an electrode system. The corresponding electronic signal produces an electrical field or an electromagnetic signal that propagates through tissue. This electric field or electromagnetic wave is then detected by an arrangement of one or more external sensor system 10 arrays 28 externally applied as a tissue site 46. In this embodiment, the frequency and waveform associated with this signal may be adjusted to enable characterization of specific phenomena. Adjustment of frequency and waveform may enable variation in the range of propagation of the signal in tissue and enable methods for localization of the measured phenomena.

Applications of the transdermal sensor system 70 may include, but are not restricted to, characterization of wound healing, monitoring of pulmonary function, monitoring of gastric function.

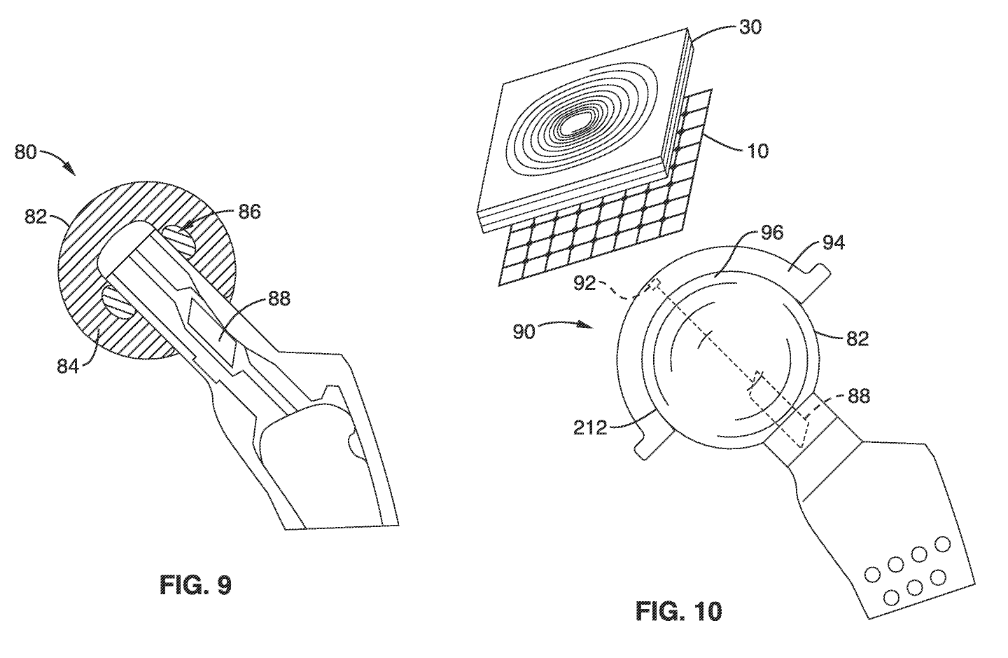

FIG. 9 illustrates a transdermal sensor system 80 for use with an orthopedic implant, e.g. total hip implant, in accordance with the present invention. Transdermal sensor system 80 provides preventive measures by enabling early detection of aforementioned mechanical issues with the implant which would otherwise have not been detected for an extended period or may have required replacement or removal of the existing implant.

The transdermal sensor system 80 that uses an interrogator 30 to provide energy to an external sensor assembly 10 and one or more intrasensor implants. In one preferred embodiment, a single intrasensor implant 88 or dual opposing intrasensor implants 84 and 86 may be positioned within the joint space on the distal femur and proximal tibia 82.

In a preferred embodiment, intrasensor implants 84, 86 or 88 may comprise an emitter element 124 (FIG. 11) that comprises a micro-scale ultrasound transducer to generate an acoustic signal to verify status of the bone-implant. The signal generated by the emitter 124 is received by the extrasensor array 10 positioned external to the body. The received data is used to generate an acoustic profile of the bone implant for determination of wear and corrosion.

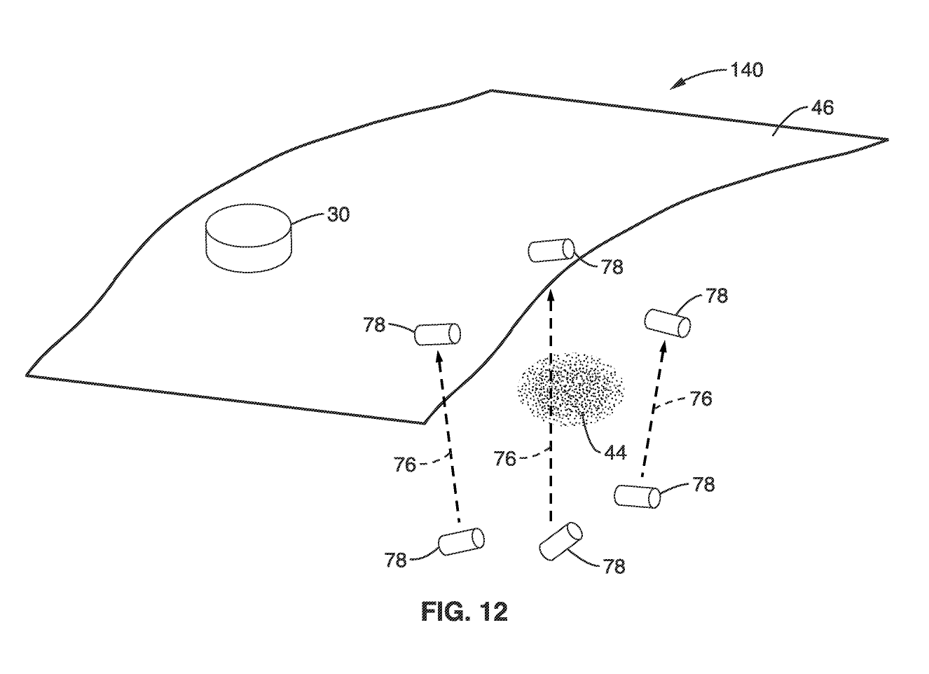

FIG. 10 illustrates a transdermal sensor system 90 having two intrasensor implants: implant 88 in the prosthetic femoral head 82, and implant 92 across the joint in the prosthetic acetabular cup 96. This configuration allows for acoustic measurement of the contact of the mating prosthetic surfaces, and any gap 96 that may have formed between them. It is also appreciated that the two-sensor configuration may be implemented as an “intersensor” system described in more detail below with respect to FIG. 12.

Additionally, an extra sensitive strain detector may be provided on the bone implant to better obtain information regarding the bone strain.

The intrasensor implants 84, 86, 88 or 92 of the prosthetic joint can be incorporated into the standard manufacturing process of hip implants or knee prostheses and implanted during total hip or knee arthroplasty.

As an additional feature, the RF or light induced energy generated by the interrogator 30 may is used to power up additional embedded sensors to measure temperature, pressure, strain or inflammation at the joint or bone tissue. The interrogator 30 may use ultrasonic wave propagation analysis and scanning acoustic microscopic techniques to map the acoustic impedance profile of the joint section. The acoustic impedance maps helps with highlighting bone resorption and bone/joint/implant remodeling on a micro structural level.

In a preferred embodiment, transdermal sensor system 70 may be configured as an optical spectroscope, having an external sensor system 10 that includes an arrangement of optical sensors, or optical emitters or a combination of optical sensors and emitters applied at the nodes 12 of the external array 28. A variety of element arrangements may be used to suit specific physiological locations and applications. Multiple intrasensor implants 72 may be employed at various locations around a region of interest as detailed in FIGS. 7 and 8, and may operate in sequence or simultaneously with data that may be combined via sensor fusion methods.

The intrasensor implant 72 elements may contain one more optical sensors or emitters that may direct and receive optical signals into and from internal tissue. The intrasensor implant 72 may also include an arrangement of multiple sensors and emitters that include optical spectroscopic filters (not shown). In addition, the intrasensor implant 72 may also include an arrangement of emitters and sensors that offer narrow solid angle of acceptance or emittance to enable an angle resolved characterization. The intrasensor implant 72 element in this configuration may include a digital control system 110 and wireless communication interface (e.g. antennas 118, 120) that enables control and coordination with external devices through a communication channel conveyed via the same radio frequency signal applied for energy transmission.

The intrasensor implant 72 elements 122, 124 may generate or receive an optical signal that is coupled to tissue via its electrode system. The corresponding external sensing system 10 elements 122, 124 may receive or transmit signals as well that are detected by intrasensor implant 72.

Applications of optical spectroscope embodiment of the transdermal sensor system 70 may include, but are not limited to, characterization of wound healing, monitoring of pulmonary function, monitoring of gastric function and monitoring of tumor growth. Optical characterization can also exploit well-known methods relying on infrared signal absorption to resolve the presence of subsurface oxyhemoglobin and deoxyhemoglobin to, for example, detect subsurface blood perfusion state in internal tissue and organs. A plurality of intrasensor implants 72 and external sensing systems 10 may be employed to enable a tomographic imaging of tissue and internal structure.

In another preferred embodiment, the transdermal sensor system 70 may be configured to comprise a passive or active acoustic spectroscope by using an arrangement of acoustic sensors or emitters or a combination of such sensors and emitters applied at the nodes 12 of the external array 28. The intrasensor implants 72 elements 122, 124 may also include an arrangement of multiple acoustic sensors and emitters.

Applications of the acoustic spectroscope embodiment of the transdermal sensor system 70 may include, but are not restricted to characterization of subsurface tissue and organ structure.

A preferred embodiment of a passive acoustic transdermal sensor system 70 may be to detect the vibration signals and acoustic emission signals that are typical of mechanical wear associated with bearing surfaces. Both external sensor system 10 and intrasensor implants 72 may contribute. This permits the detection of wear indication associated with biomedical implant devices whether associated with joints (knee or hip), dental implants, or the like. Those skilled in the art will be familiar with the means of applying condition based monitoring (CBM) principles for this detection [Williams 2002].

3. InterSensor System

FIGS. 12 through 15 illustrate the “Intersensor” system of the present invention. For purposes of this description, an “InterSensor” is defined as an internal sensing implant or implants that receive and or transmit physiological signals entirely within human or animal tissue. The internal sensing implants of the “Intersensor” system are externally-interrogated to receive/transmit data relating to instructions for performing measurements and data relating to previously performed internal measurements, in addition to providing operating energy for the internal sensing implant(s).

Referring now to FIG. 12, an intersensor system 140 in accordance with the present invention includes one or more internal sensing implants 78 disposed internally in the body adjacent an anatomical region of interest 44 below the skin surface 46. Internal sensing implants 78 receive and or transmit physiological signals entirely within human or animal tissue, and derive operating energy primarily or entirely from the receipt of externally applied electromagnetic signals (e.g. radio frequency (RF) energy) from interrogator 30 that is attached to or located above the skin 46.

As shown in FIG. 12, the internal sensing implants 78 are configured in a transmissive mode wherein one or more internal sensing implants 78 transmit a signal 76 to be received by one or more additional internal sensing implants 78. Signal 76 is configured to be transmitted through tissue to characterize at least one physiological aspect of the tissue. In this configuration, some of the internal sensing implants 78 may be configured with just an emitter element 124 to transmit a signal, whereas others may be equipped with only a sensor element 122 to receive a signal.

Internal sensing implants 78 may also be implemented in a passive mode for receiving physiological signals emitted from an internal region of interest 44 (similar to signals 48 of FIG. 3, except that the signals emanate and are received entirely subcutaneously). In this configuration, the internal sensing implants 78 may be configured with only a sensor element 122 to receive a signal.

Internal sensing implants 78 may also be implemented in a reflective mode for transmitting signals 40 at or around an internal region of interest 44, and receiving reflected signals 42 that contain data relating to a physiological characteristic of the internal region of interest 44 (similar to signals 40, 42 of FIG. 2, except that the signals are transmitted and are received entirely subcutaneously). In this configuration, some of the internal sensing implants 78 may be configured with configured with both an emitter element 124 to transmit a signal and a sensor element 122 to receive a signal.

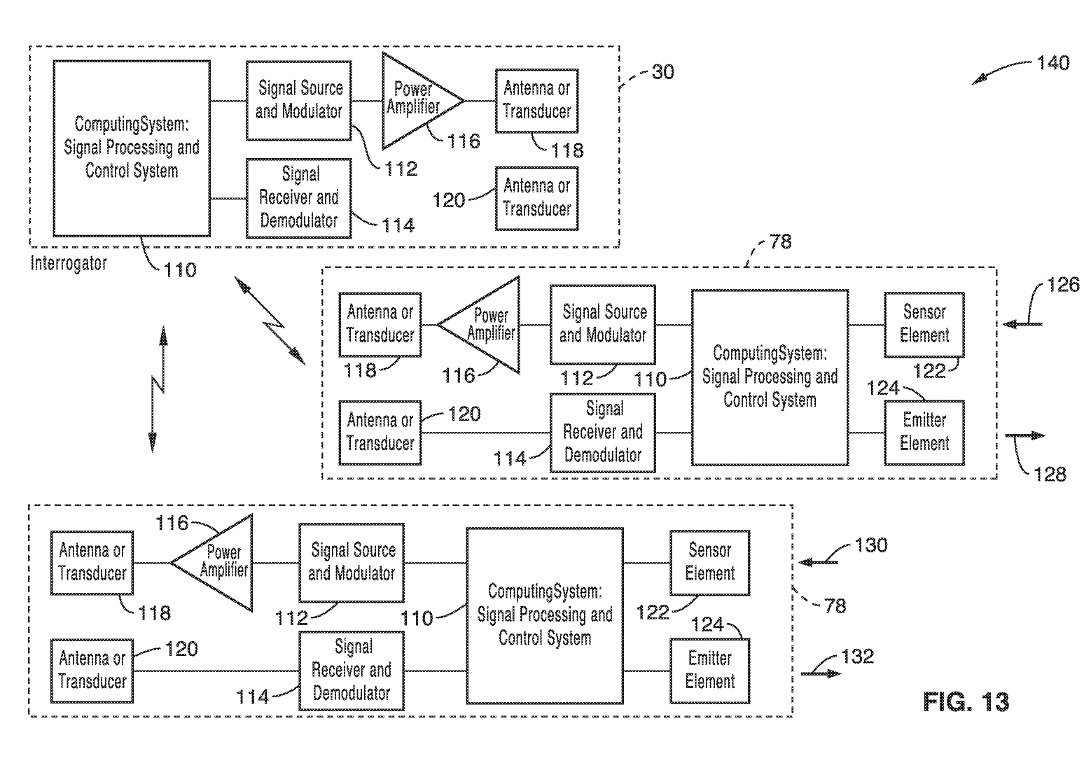

FIG. 13 illustrates a schematic diagram of the primary components of intersensor system 140 in accordance with the present invention. Intersensor system 140 includes an interrogator 30 that is configured to communicate with and provide power to one or more intrasensor implants 78. The interrogator 30 provides the source energy (e.g. radio frequency (RF) electromagnetic signals) and communication for operation of the one or more internal sensing implants 78. The interrogator 30 is configured to provide time synchronized and time and event coordinated operation of the internal sensing implants 78.

As shown in FIG. 13, the interrogator 30 includes a processor 110 for commanding and controlling the operation of internal sensing implant 78 elements according to a sequence of operations upon a set of programming instructions stored within memory on the interrogator 30 (e.g. via board 36 shown in the interrogator 30 of FIG. 1), or provided to the interrogator from an outside source. The processor 110 is also configured to receive, process, and store information from internal sensing implant 78.

The interrogator 30 further includes a signal generator and modulator 112 to permit the transmission of data. A power amplifier 116 amplifies the modulated signal, which is then transmitted via antenna or transducer 118 for reception by the internal sensing implant 78.

In a preferred embodiment, the signal generator and modulator 112 are configured to generate a radio frequency (RF) electromagnetic signals. In such configuration, the antenna 118 may comprise a coil antenna 32 (as shown in shown in interrogator 30 of FIG. 1), configured to generate the radio frequency signal.

The interrogator 30 further includes an antenna or transducer 120 to receive communication transmissions from the internal sensing implants 78. The antenna 120 is coupled to a signal receiver and demodulator 114 to demodulate the radio frequency signal so as to permit the reception and recovery of data for processor 110. In an alternative embodiment, it is possible that only one antenna (e.g. antenna 118) is used for both transmission and reception of signals.

Each internal sensing implant 78 comprises a processor 110 for commanding emitter element 124 and receiving data from sensor element 122 with regard to their sequence of operations to affect the desired physiological measurements within the target tissue 44. For example, the emitter element 124 may emit a signal 128 into and through an adjacent region of tissue. In reflective operation the emitted signal may be reflected back as signal 126 to be received by sensor element 122.

Alternatively, in a transmissive operation, the emitted signal 128 is received as incoming signal 130 by sensor element 122 of another internal sensing implant 78. It is also appreciated that the internal sensing implant 78 may only comprise one of either an emitter element 124 or sensor element 122 for one-way transmissive communication with neighboring internal sensing implants 78.

The internal sensing implant 78 is capable of receiving data, information or commands from interrogator 30 via antenna or transducer 120. This data is received and demodulated at 114 to rectify the signal properly to derive potentials that may enable operation of microelectronic circuits.

The internal sensing implant 78 further includes a signal generator and modulator 112 to permit the transmission of data (e.g. acquired physiological data) back to the interrogator 30. A power amplifier 116 amplifies the modulated signal, which is then transmitted via antenna or transducer 118 for reception by the interrogator 30.

Further, each of the internal sensing implants 78 comprise a means (e.g. antenna/transducer 118) to produce an electromagnetic signal comprising a data communication carrier signal that may be received by the interrogator 30 for the purposes of conveying information from the internal sensing implants 78. This information may include data describing the signals associated with sensor and emitter elements 122 and 124.

The data communication carrier signal described above preferably comprises an electromagnetic propagating wave as familiar to those skilled in the art of RFID technology. However, it is appreciated that the data communication carrier may be an optical, acoustic, or other signal that provides an adequately reliable data communication channel. This data communication carrier signal may also convey energy as required or operation of the internal sensing implant 78. For example, where an electromagnetic propagating wave is replaced by optical, acoustic, or other signals, then appropriate transducers for respectively, optical (e.g. photodiode emitters and sensors) or acoustic (e.g. ultrasound emitters and sensors), or other signals will vary accordingly for respective receipt of signals and conveyance of necessary energy.

The interrogator 30 enables the communication of data from the interrogator computing system or processor 110 to the computing systems of the internal sensing implants 78. This occurs via the process of first generating data, modulation of this data onto a data communication carrier signal, introduction of a power amplification step, and finally the emission of this data from an antenna or appropriate transducer and its propagation to the internal sensing implant 78. At the internal sensing implant 78, this data communication carrier is received, demodulated and made available as data to the computing system that is part of the respective internal sensing implant 78. Finally, the data transmitted between interrogator 30 and internal sensing implant 78 may include sensor measurement data associated with physiological signals (including those associated with bioelectric impedance, optical spectroscopic, or acoustic spectroscopic). The data transmitted between interrogator 30 and internal sensing implant 78 may also include program sequence instructions intended to be applied by the computing system of the respective interrogator 30 and internal sensing implant 78 for control of both the function of emitter and sensor elements.

Finally, the internal sensing implants 78 include emitter and sensor elements 122, 124 that generate and receive physiological signals, including those associated with bioelectric impedance, optical spectroscopic, or acoustic spectroscopic. These signals propagate between internal sensing implants 78, or are reflected or transmitted to sensing implant 78 from neighboring tissue.

In one preferred embodiment, multiple intrasensor implants 72 operate in sequence or simultaneously with data that may be combined via sensor fusion methods for inference of internal organ state.

The implant 78 elements 122, 124 may include a dedicated digital control system and wireless communication interface that enables control and coordination with the interrogator 30 through a communication channel conveyed via the same radio frequency signal applied for energy transmission, or a separate channel. This communication channel may exploit means that are familiar to those skilled in the art of RFID technology.

The implant 78 emitting elements 124 may generate an electronic signal that is coupled to tissue via an electrode system. The corresponding electronic signal produces an electrical field or an electromagnetic signal that propagates through tissue. This electric field or electromagnetic wave is then detected by an arrangement of one or more. In this embodiment, the frequency and waveform associated with this signal may be adjusted to enable characterization of specific phenomena. Adjustment of frequency and waveform may enable variation in the range of propagation of the signal in tissue and enable methods for localization of the measured phenomena.

Applications of the intersensor system 140 may include, but are not limited to, characterization of wound healing, monitoring of pulmonary function, and monitoring of gastric function.

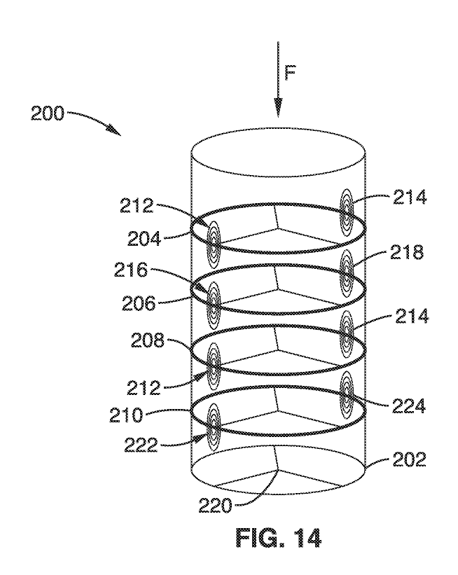

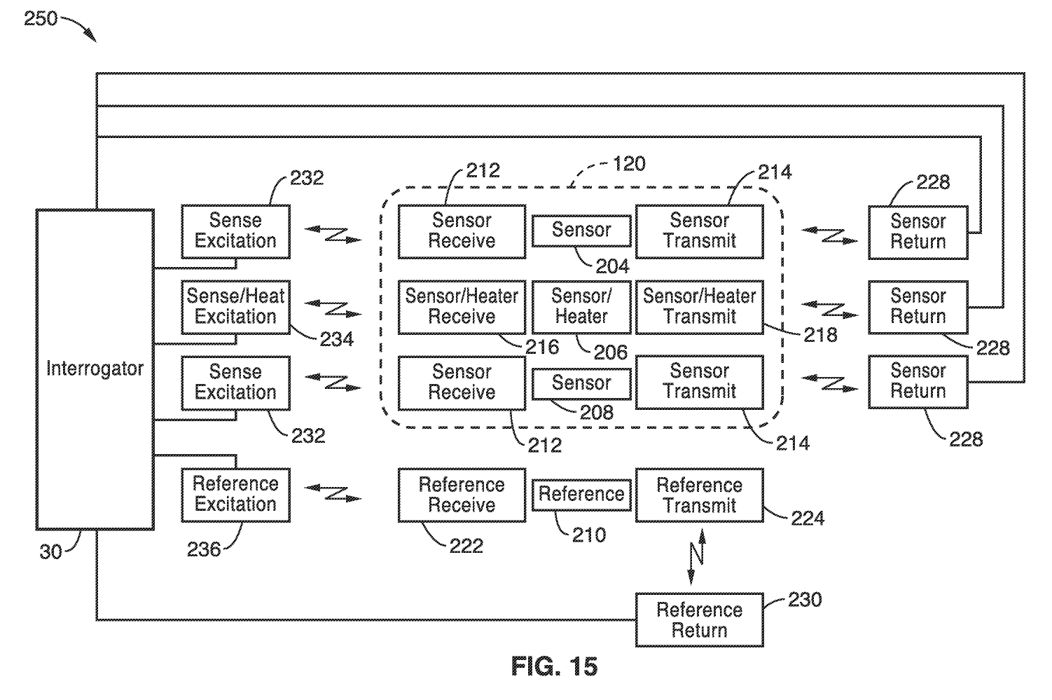

In one embodiment shown in FIGS. 14 and 15 an intersensor system 200 may comprise a pulmonary stent containing wireless in situ sensors for monitoring airflow or cardiothoracic stent containing wireless in situ sensors for monitoring blood flow.

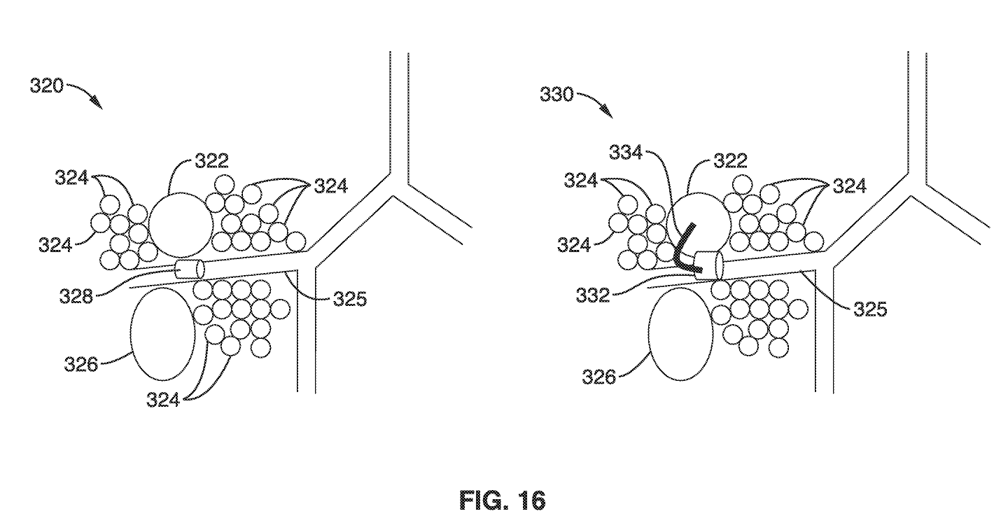

Intersensor system 200 comprises a stent structure 202 that is sized and configured to be delivered into an internal lumen (e.g. air passage 325 shown in FIG. 16) and expanded to conform to the lumen 325 internal diameter. Stent structure 202 is equipped with multiple receive, transmit, and reference inductors/sensors for the acquisition and transmission of data relating to a physiological condition (e.g. flowrate F) of the lumen 325. The receive inductors/antennas 212, and 216 receive radiofrequency (RF) and/or light energy from the interrogator 30 (FIG. 15) and supply this energy (and operation commands) to corresponding sensing elements 204, 206, and 208. Sensing elements 204, 206, and 208 may include sensors for measurement of temperature, strain, or position. Sensing elements can then enable measurements of mass flow, system strain, or the position of a vane or valve 220 on the stent 202. Sensing measurement circuits within the device may provide measurements of resistance (for example for temperature or strain measurements), position (for example of a vane or valve), or other parameters. The receive inductors/sensors 212, and 216 may also be accompanied with magnetic elements to permit actuation of a vane or valve 202 for an active (vs. passive) stent.

In a preferred embodiment, the stent comprises a heating element 216 that induces heat into flow F. The upstream temperature is measured at sensor 204, and downstream temperature is measured as sensor 208 to detect a temperature difference measurement in the flow resulting from the presence and operation of the heater 206. This temperature difference through proper calibration may then be used to determine flow rate F according to methods familiar to those skilled in the art of thermal mass flow measurement methods.

The stent 202 further includes transmission antennas 214, and 218 for transmitting the acquired physiological data back to the interrogator for retrieval.

A reference sensor 210 along with reference excitation 206, reference return 220, reference receive 222 and reference transmit 224 comprise a means of system calibration. Here the reference sensor is not responsive to environmental phenomena. Thus, its response provides a means to determine the variation in system response resulting from variables in the properties of the interrogator and other elements and as well as their relative position.

The interrogator 30 may provide capabilities such as delivery and feedback control of RF and light energy; measurement of return signals; computation for determining mass air flow F via thermal heat transfer methods, mass air flow via vane 220 deflection position measurement methods, valve 220 state via valve deflection position measurement methods that rely on either strain or capacitance measurements via either direct measurement or via detection of the resonance frequency of passive circuits incorporating the capacitance; delivery and control of energy required for opening, closing, and regulating valve 220 state, reference calibration etc.

The reference calibration functionality and elements address problems associated with uncertainty in location of the stent, and its potential impact on operation (e.g. disturbance to flow by presence in the flow) is removed through the architecture of the stent and interrogator software (e.g. calibration of the stent data). The elements receive the same RF energy flux, and then return, via the transmit function, a calibrated signal. Together, the reference elements 210 provide a means to eliminate the effects of location uncertainty. Further, these methods ensure that operation will occur only under the presence of a properly aligned interrogator 30 and an interrogator 30 that matches required characteristics.

FIG. 15 illustrates a schematic diagram of the components of the stent 200 and interrogator 30.

The stent system 200 could be used in place of current stents used in bronchoscopic lung volume reduction (BLVR) in COPD patients. Additionally, the stent 200 could be inserted in patients deemed to have a high risk of lung tissue collapse for the purposes of monitoring lung function.

FIG. 16 illustrates an in situ intersensor system 320 with internal sensor 328, which may comprise stent 200 in accordance with the present invention to measure flow rate through a lumen 325 of the lung. The illustration on the right shows stunted flow of the airway via valve 334.

It is also appreciated that by inclusion of a second intersensor 328 (not shown) transmissive signals may be sent out into neighboring tissues 322, 324, and 326 to obtain physiological data with respect to said tissues.

The addition of sensor technology to stents for bronchoscopic placement has the potential to transform the treatment emphysema, as it will decrease the risk of delay in complication determination and it will track progress, which is currently limited due to the masking affect that is witnessed in global measures of lung function.

The system of the present invention offers a safe and convenient interrogation method for effectively guiding COPD rehabilitation and treatment that has not been previously available ND provides on-demand feedback on the status of COPD devices absent a visit to the clinic. Moreover, the present invention can be used to assess functional derangements occurring in the context of altered symptoms, and to better marry physiologic information with symptoms in a way that cannot otherwise be captured. The classical outcomes measures used to monitor patients with endobronchial devices are measures of airflow, lung volumes and exercise testing, all of which require specialized equipment

It is anticipated that the successful functioning of endobronchial valves will result in a decrease in content of oxygen and an increase in content of carbon dioxide in the non-conducting central airways relative to pre-intervention. Additionally, the therapeutic effects of these non-surgical airway stents can be measured by alterations in airflow resulting from improved FVC.

One major implication of this sensor-enhanced paradigm of the present invention is the ability to better manage the individual patient. In addition, alterations in signal content will be integrated with the activity level of the patient and standardized assessments of symptoms. By maintaining the data collected in these patients in a signal database, pattern classification, search, and pattern matching algorithms can be developed to better map symptoms with fluctuations in respiratory function. This approach is not limited to the specific condition of emphysema, but may have broad application in all forms of COPD and even reactive airways diseases, can be used to presage COPD exacerbations, which are a major cause of morbidity and mortality in the COPD patient.

The intersensor system embodiments disclosed above may be implemented as optical and passive and active acoustical spectroscopes by varying the structure of the sensor and emitter elements antennas and operational software, as explained above for the intrasensor embodiments.

While the embodiments disclosed in FIGS. 1-16 are primarily directed to diagnostic system and methods, it is appreciated that the

Embodiments of the present invention are described with reference to flowchart illustrations of methods and systems according to embodiments of the invention. These methods and systems can also be implemented as computer program products. In this regard, each block or step of a flowchart, and combinations of blocks (and/or steps) in a flowchart, can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts support combinations of means for performing the specified functions, combinations of steps for performing the specified functions, and computer program instructions, such as embodied in computer-readable program code logic means, for performing the specified functions. It will also be understood that each block of the flowchart illustrations, and combinations of blocks in the flowchart illustrations, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s).

From the discussion above it will be appreciated that the invention can be embodied in various ways, including the following:

1. An interrogatable external sensor system for acquiring one or more biological characteristics of a surface or internal tissue region of a body of a patient, comprising: a sensor array; an interrogator configured to transmit energy in the form of an electromagnetic waveform; said sensor array comprising: a substrate configured to be positioned external to and proximal to the patient's body; a plurality of sensor elements coupled to the substrate; a processor coupled to the substrate and connected to the plurality of sensor elements; said processor configured to communicate with at least one of the sensors elements in the array; wherein the sensor elements are configured to emit or receive a physiological signal through the internal tissue region or at a surface tissue region; wherein the physiological signal comprises at least one physiological characteristic of the surface or internal tissue region; and an antenna coupled to the array; wherein the antenna is responsive to electromagnetic energy transmitted from the interrogator; wherein the electromagnetic energy powers the array with sufficient energy to power the emission or reception of the physiological signal through at least one of the sensor elements.

2. The system of embodiment 1: wherein the electromagnetic energy comprises RF energy; wherein the sensor elements comprise a plurality of sensor or emitter electrodes; and wherein the antenna comprises an RF coil configured to inductively power at least one of the electrodes.

3. The system of embodiment 1: wherein the electromagnetic energy comprises the sole source of power to the array.

4. The system of embodiment 1, wherein the electromagnetic waveform comprises a data signal; and wherein the data signal comprises instructions readable by said processor for controlling the one or more elements.

5. The system of embodiment 1: wherein the electromagnetic energy comprises an optical waveform; wherein the sensor elements comprise a plurality of optical sensors or emitters; and wherein the antenna comprises an optical receiver configured to inductively power at least one of the optical sensors or emitters.

6. The system of embodiment 1: wherein the electromagnetic energy comprises an acoustic waveform; wherein the sensor elements comprise a plurality of acoustic transducers; and wherein the antenna comprises a transducer configured to inductively power at least one of the acoustic transducers.

7. The system of embodiment 1, wherein said sensors elements are selected from the group of sensors consisting essentially of temperature sensors, moisture sensors, pressure sensors, bioelectric impedance sensors, electrical capacitance sensors, spectroscopic sensors, and optical sensors.

8. The system of embodiment 4, wherein the array further comprises a signal demodulator to demodulate the electromagnetic signal for processing by the processor.

9. The system of embodiment 8, wherein the array further comprises a signal modulator for transmitting a return data signal relating to said physiological characteristic from the array to the interrogator.

10. The system of embodiment 1, wherein the sensor elements are disposed at intersections of row and column transmission lines; and wherein said transmission lines are coupled to said processor for individual control of the sensor elements.

11. The system of embodiment 1, wherein the array is configured to comprise at least one emitter element configured to emit a signal into the internal tissue region and at least on sensor element configured to receive a reflected signal from said tissue region; wherein the reflected signal comprises at least one physiological characteristic of said tissue region.

12. The system of embodiment 1, wherein the sensor array comprises a first sensor array, the system further comprising: a second array of sensor elements; the second array configured to be positioned external to and adjacent the patient's skin; the second array comprising: a plurality of sensor elements; and a processor connected to the plurality of sensor elements; said processor configured to communicate with at least one of the sensors elements in the array; wherein at least one sensor element of the second array is configured to emit a transmissive signal through the internal tissue region for reception by at least one sensor element in the first sensor array; wherein physiological signal comprises at least one physiological characteristic of the internal tissue region.

13. The system of embodiment 12, further comprising a second antenna coupled to the second array; wherein the second antenna is responsive to electromagnetic energy transmitted from the interrogator; and wherein the electromagnetic energy powers the second array with sufficient energy to power the emission of the transmitted signal through the internal tissue region to the first array.

14. The system of embodiment 1, further comprising: an implant disposed at or near the internal tissue region; wherein the implant comprises at least one sensor element configured to emit a transmissive signal through the internal tissue region for reception by at least one sensor element in the second sensor array.

15. The system of embodiment 14, further comprising a second antenna coupled to the implant; wherein the second antenna is responsive to electromagnetic energy transmitted from the interrogator; and wherein the electromagnetic energy powers the second antenna with sufficient energy to power the emission of the transmitted signal through the internal tissue region to the first array.

16. A method for acquiring one or more biological characteristics of a surface or internal tissue region of a patient, comprising: positioning a sensor array external to and adjacent to a region of the patient's skin; wherein the array comprises a plurality of sensor elements connected to a processor; positioning an interrogator in proximity to said array; the interrogator configured to transmit energy in the form of an electromagnetic waveform; transmitting an electromagnetic signal from the interrogator; receiving the electromagnetic signal via an antenna coupled to the array; inductively powering the array via the electromagnetic signal; and instructing the array via the electromagnetic signal to emit or receive a physiological signal through the internal tissue region or at a surface tissue region; wherein the physiological signal comprises at least one physiological characteristic of the surface or internal tissue region.

17. The method of embodiment 16: wherein the electromagnetic energy comprises RF energy and the antenna comprises an RF coil; wherein the array comprises a plurality of sensor or emitter electrodes; and wherein inductively powering the array comprises powering the RF coil with sufficient energy to power at least one of the sensor or emitter electrodes.

18. The method of embodiment 16: wherein the electromagnetic energy comprises the sole source of power to the array.

19. The method of embodiment 16, wherein the electromagnetic signal comprises a data signal; and wherein instructing the array comprises reading the data signal with said processor and operating at least one sensor element in the array based on one or more instructions is said data signal.

20. The method of embodiment 16, wherein said sensor array comprises sensors are selected from the group of sensors consisting essentially of temperature sensors, moisture sensors, pressure sensors, bioelectric impedance sensors, electrical capacitance sensors, spectroscopic sensors, and optical sensors.

21. The method of embodiment 19, further comprising: demodulating the electromagnetic signal for processing by the processor.

22. The method of embodiment 21, further comprising: modulating a return signal relating to said physiological characteristic for transmission to the interrogator.

23. The method of embodiment 16, wherein the sensor elements are disposed at intersections of row and column transmission lines; and wherein said transmission lines are coupled to said processor for individual control of the sensor elements.

24. The method of embodiment 16, further comprising: emitting a signal into the internal tissue region; and receiving a reflected signal from said tissue region; wherein the reflected signal comprises at least one physiological characteristic of said tissue region.

25. The method of embodiment 16, wherein the sensor array comprises a first sensor array, the method further comprising: positioning a sensor array external to and adjacent to a region of the patient's skin; emitting a transmissive physiological signal from the second sensor array through the internal tissue region for reception by the first sensor array; wherein the physiological signal comprises at least one physiological characteristic of the internal tissue region.

26. The method of embodiment 25, further comprising a second antenna coupled to the second array; wherein the second antenna is responsive to electromagnetic energy transmitted from the interrogator; and powering the second array with sufficient energy to power the emission of the transmitted physiological signal through the internal tissue region to the first array.

27. The method of embodiment 16, further comprising: delivering an implant at or near the internal tissue region; emitting a transmissive physiological signal from the implant through the internal tissue region for reception by the second sensor array.

28. The method of embodiment 27, wherein the implant comprises a second antenna responsive to electromagnetic energy transmitted from the interrogator, the method further comprising; powering the second antenna with sufficient energy to power the emission of the transmitted physiological signal through the internal tissue region to the first array.

29. A transdermal sensor system for acquiring one or more biological characteristics of an internal tissue region of a patient, comprising: an interrogator configured to transmit energy in the form of an electromagnetic waveform; an external sensor array; an implant disposed at or near the internal tissue region; wherein the implant comprises at least one internal sensor element configured to exchange a transmissive physiological signal through the internal tissue region with the external sensor array; wherein the physiological signal comprises at least one physiological characteristic of the internal tissue region; wherein the implant comprises an internal antenna responsive to electromagnetic energy transmitted from the interrogator; and wherein the electromagnetic energy powers the implant with sufficient energy to power the exchange of the physiological signal through the at least one internal sensor element.

30. The system of embodiment 29: wherein said external sensor array comprises: a substrate configured to be positioned external to and adjacent the patient's skin; a plurality of external sensor elements coupled to the substrate; and an array processor coupled to the substrate and connected to the plurality of external sensor elements; said array processor configured to communicate with at least one of the external sensor elements in the array; wherein the external sensor elements are configured to emit or receive the physiological signal; an external antenna coupled to the array; wherein the external antenna is responsive to electromagnetic energy transmitted from the interrogator; and wherein the electromagnetic energy powers the array with sufficient energy to power the exchange of the transmissive physiological signal with the implant.

31. The system of embodiment 30: wherein the at least one internal sensor element comprises an emitter; wherein at least one of the external sensor elements comprises a sensor; and wherein the implant is configured to emit the transmissive physiological signal through the internal tissue region from the emitter for reception by the sensor of the external sensor array.

32. The system of embodiment 30: wherein the at least one internal sensor element comprises a sensor; wherein at least one of the external sensor elements comprises an emitter; and wherein the external sensor array is configured to emit the transmissive physiological signal through the internal tissue region from the emitter for reception by the sensor of the implant.

33. The system of embodiment 30: wherein the electromagnetic energy comprises RF energy; wherein the external and internal sensor elements comprise sensor or emitter electrodes; and wherein the external and internal antennas comprise RF coils configured to inductively power the sensor or emitter electrodes.

34. The system of embodiment 30: wherein the electromagnetic energy comprises the sole source of power to the array.

35. The system of embodiment 30: wherein the implant comprises an implant processor coupled to the at least one sensor element; said implant processor configured to communicate with the at least one sensor element; wherein the electromagnetic waveform comprises a data signal; and wherein the data signal comprises instructions readable by said implant processor and said array processor for controlling at least one sensor element.

36. The system of embodiment 30: wherein the electromagnetic energy comprises an optical waveform; wherein the sensor elements comprise a plurality of optical sensors or emitters; and wherein the external and internal antennas comprise an optical receiver configured to inductively power at least one of the optical sensors or emitters.

37. The system of embodiment 30: wherein the electromagnetic energy comprises an acoustic waveform; wherein the sensor elements comprise a plurality of acoustic transducers; and wherein the external and internal antennas comprise a transducer configured to inductively power at least one of the acoustic transducers.

38. The system of embodiment 29, wherein said sensors elements are selected from the group of sensors consisting essentially of temperature sensors, moisture sensors, pressure sensors, bioelectric impedance sensors, electrical capacitance sensors, spectroscopic sensors, and optical sensors.

39. The system of embodiment 35, wherein the external array and implant each further comprise a signal demodulator to demodulate the electromagnetic signal.

40. The system of embodiment 39, wherein the external array and implant each further comprise a signal modulator for transmitting a return data signal relating to said physiological characteristic from either the external array or the implant to the interrogator.

41. The system of embodiment 29, wherein the implant is disposed on an internally implanted prosthetic device; wherein the internal sensor element is configured to exchange a transmissive physiological signal through at least a portion of the internally implanted prosthetic device with the external sensor array; and wherein the a transmissive physiological signal relates to a physiological characteristic of the internally implanted prosthetic device.

42. A method for acquiring one or more biological characteristics of an internal tissue region of a patient, comprising: positioning a sensor array external to and adjacent to a region of the patient's skin; delivering an implant to a location at or near the internal tissue region; positioning an interrogator in proximity to said array; the interrogator configured to transmit energy in the form of an electromagnetic waveform; wherein the implant comprises an internal antenna responsive to electromagnetic energy transmitted from the interrogator; transmitting an electromagnetic signal from the interrogator; receiving the electromagnetic signal via the internal antenna; inductively powering the implant via the electromagnetic signal; and instructing the implant via the electromagnetic signal to exchange a physiological signal with the external array through at least a portion of the internal tissue region; wherein the physiological signal comprises at least one physiological characteristic of the internal tissue region.

43. The method of embodiment 42, wherein the implant comprises at least one internal sensor element configured to exchange a transmissive physiological signal through the internal tissue region with the external sensor array; wherein the implant comprises an internal antenna responsive to electromagnetic energy transmitted from the interrogator; and wherein the electromagnetic energy powers the implant with sufficient energy to power the exchange of the physiological signal through the at least one internal sensor element.

44. The method of embodiment 43: wherein said external sensor array comprises a plurality of external sensor elements configured to emit or receive the physiological signal, an external antenna coupled to the array, and an array processor configured to communicate the antenna and at least one of the external sensor elements in the array; wherein the external antenna is responsive to electromagnetic energy transmitted from the interrogator; and wherein the electromagnetic energy powers the array with sufficient energy to power the exchange of the transmissive physiological signal with the implant.

45. The method of embodiment 42: wherein exchanging the physiological signal comprises emitting the transmissive physiological signal from the implant through the internal tissue region for reception by the external sensor array.

46. The method of embodiment 42: wherein exchanging the physiological signal comprises emitting the transmissive physiological signal from the external sensor array through the internal tissue region for reception by the implant.

47. The method of embodiment 44: wherein the electromagnetic energy comprises RF energy; wherein the external and internal sensor elements comprise sensor or emitter electrodes; and wherein inductively powering the implant comprises powering the external and internal antennas to inductively power the sensor or emitter electrodes.

48. The method of embodiment 44, wherein the electromagnetic signal comprises a data signal and the implant comprises an implant processor coupled to the at least one internal sensor element; and wherein instructing the implant comprises reading the data signal with said implant processor and operating the at least one sensor element based on one or more instructions in said data signal.

49. The method of embodiment 42, wherein said implant and external sensor array are selected from a group of sensors consisting essentially of temperature sensors, moisture sensors, pressure sensors, bioelectric impedance sensors, electrical capacitance sensors, spectroscopic sensors, and optical sensors.

50. The method of embodiment 48, further comprising: demodulating the electromagnetic signal for processing by the implant processor.

51. The method of embodiment 48, further comprising: modulating a return signal relating to said physiological characteristic for transmission from the implant to the interrogator.

52. The method of embodiment 48, further comprising: modulating a return signal relating to said physiological characteristic for transmission from the external sensor array to the interrogator.

53. The method of embodiment 42, further comprising: delivering a second implant at or near the internal tissue region; exchanging a second transmissive physiological signal through the internal tissue region with the external sensor array.

54. An interrogatable sensor system for acquiring one or more biological characteristics of an internal tissue region of a patient, comprising: an interrogator configured to be positioned at a location external to the body of the patient and transmit energy in the form of an electromagnetic waveform; a first implant configured to be disposed at or near the internal tissue region; wherein the first implant comprises a sensor element configured to receive a physiological signal through at least a portion of the internal tissue region; wherein the physiological signal emanating within the body of the patient and comprising at least one physiological characteristic of the internal tissue region; wherein the first implant comprises an antenna responsive to electromagnetic energy transmitted from the interrogator; and wherein the electromagnetic energy powers the implant with sufficient energy to power the receipt of the physiological signal through the sensor element.

55. The system of embodiment 54, wherein the first implant further comprises an emitter element coupled to the antenna; and wherein the emitter element is configured to emit a physiological signal into at least a portion of the internal tissue region; physiological signal comprising at least one physiological characteristic of the internal tissue region.

56. The system of embodiment 55, wherein the sensor element is configured to receive a reflected signal from the internal tissue region; wherein the reflected signal emanates from the emitter.

57. The system of embodiment 55: wherein the electromagnetic energy comprises RF energy; wherein the sensor element and emitter element comprise sensor or emitter electrodes; and wherein the antenna comprises an RF coil configured to inductively power at least one of the electrodes.

58. The system of embodiment 54: wherein the electromagnetic energy comprises the sole source of power to the array.

59. The system of embodiment 54: wherein the first implant further comprises a first processor coupled to the internal antenna and sensor element; wherein the electromagnetic waveform comprises a data signal; and wherein the data signal comprises instructions readable by said first processor for controlling the sensor elements.

60. The system of embodiment 55: wherein the electromagnetic energy comprises an optical waveform; wherein the sensor element and emitter element comprise optical sensors or emitters; and wherein the internal antenna comprises an optical receiver configured to inductively power at least one of the optical sensor or emitter.

61. The system of embodiment 55: wherein the electromagnetic energy comprises an acoustic waveform; wherein the sensor element and emitter element comprise an acoustic transducer; and wherein the internal antenna comprises a transducer configured to inductively power at least one of the acoustic transducers.

62. The system of embodiment 54, wherein said sensor element is selected from the group of sensors consisting essentially of temperature sensors, moisture sensors, pressure sensors, bioelectric impedance sensors, electrical capacitance sensors, spectroscopic sensors, and optical sensors.

63. The system of embodiment 59, wherein the first implant further comprises a signal demodulator to demodulate the electromagnetic signal for processing by the first processor.

64. The system of embodiment 59, wherein the first implant further comprises a signal modulator for transmitting a return data signal relating to said physiological characteristic from the array to the interrogator.

65. The system of embodiment 59, further comprising: a second implant configured to be disposed at or near the internal tissue region; wherein the second implant comprises an emitter element configured to emit a physiological signal through at least a portion of the internal tissue region; wherein the physiological signal comprises at least one physiological characteristic of the internal tissue region; wherein the second implant comprises an antenna responsive to electromagnetic energy transmitted from the interrogator; and wherein the electromagnetic energy powers the second implant with sufficient energy to power the transmission of the physiological signal through at least a portion of the internal tissue region to be received by the first implant.

66. The system of embodiment 54, wherein the first implant further comprises: a stent structure configured to be delivered to a location within the body of the patient; the stent structure comprising a central channel configured to allow fluid communication therethrough; wherein the sensor element comprises a first sensor element configured to receive a first physiological signal relating to the fluid communication through the stent; the stent structure configured to house the first sensor element and a second sensor element; the sensor configured to receive a second physiological signal relating to the fluid communication through the stent.

67. The system of embodiment 66, wherein the stent further comprises a heating element disposed between the first sensor element and the second sensor element; wherein first sensor element is configured to receive a first temperature measurement and the second sensor element is configured to receive a second temperature measurement; wherein the first and second measurements relate to a flowrate of the fluid communication through the stent.

68. A method for acquiring one or more biological characteristics of an internal tissue region of a patient, comprising: positioning an interrogator at a location external to the body of the patient; the interrogator configured to transmit energy in the form of an electromagnetic waveform; delivering a first implant to a location at or near the internal tissue region; wherein the first implant comprises a sensor element configured to receive a physiological signal through at least a portion of the internal tissue region; wherein the first implant comprises an antenna responsive to electromagnetic energy transmitted from the interrogator; transmitting an electromagnetic signal from the interrogator; receiving the electromagnetic signal via the antenna; inductively powering the first implant via the electromagnetic signal; and instructing the implant via the electromagnetic receive a physiological signal emanating within the body of the patient and comprising at least one physiological characteristic of the internal tissue region; wherein the electromagnetic energy powers the implant with sufficient energy to power the receipt of the physiological signal through the sensor element.

69. The method of embodiment 68, wherein the first implant further comprises an emitter element coupled to the antenna, the method further comprising: instructing the first implant via the electromagnetic signal to emit a physiological signal into the body of the patient from the emitter element; wherein the electromagnetic energy powers the implant with sufficient energy to power the transmission of the physiological signal.

70. The method of embodiment 69, wherein the sensor element is configured to receive a reflected signal from the internal tissue region; the reflected signal emanating from the emitter.

71. The method of embodiment 69: wherein the electromagnetic energy comprises RF energy; wherein the sensor element and emitter element comprise sensor or emitter electrodes; and wherein inductively powering the implant comprises powering the antenna to inductively power at least one of the electrodes.

72. The method of embodiment 68: wherein the electromagnetic energy comprises the sole source of power to the array.

73. The method of embodiment 68: wherein the first implant further comprises a first processor coupled to the antenna and sensor element; wherein the electromagnetic waveform comprises a data signal; and wherein instructing the implant comprises reading the data signal with said first processor and operating the sensor element based on one or more instructions in said data signal.

74. The method of embodiment 68, wherein said sensor is selected from a group of sensors consisting essentially of temperature sensors, moisture sensors, pressure sensors, bioelectric impedance sensors, electrical capacitance sensors, spectroscopic sensors, and optical sensors.

75. The method of embodiment 73, further comprising: demodulating the electromagnetic signal for processing by the first processor.

76. The method of embodiment 73, further comprising: modulating a return signal relating to said physiological characteristic for transmission from the implant to the interrogator.

77. The method of embodiment 68, further comprising: delivering a second implant at or near the internal tissue region; wherein the second implant comprises an emitter element configured to emit a physiological signal through at least a portion of the internal tissue region; wherein the physiological signal comprises at least one physiological characteristic of the internal tissue region; wherein the second implant comprises an antenna responsive to electromagnetic energy transmitted from the interrogator; and powering the second implant via the electromagnetic energy sufficiently to power the transmission of the physiological signal through at least a portion of the internal tissue region to be received by the first implant.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. An interrogatable external sensor system for acquiring one or more biological characteristics of a surface or internal tissue region of a body of a patient, comprising:
a sensor array;
an interrogator configured to transmit energy in the form of an electromagnetic waveform;
said sensor array comprising:
a substrate configured to be positioned external to and proximal to the patient's body;
a plurality of sensor elements coupled to the substrate;
a processor coupled to the substrate and connected to the plurality of sensor elements;
said processor configured to communicate with at least one of the sensors elements in the array;
wherein the sensor elements are configured to emit or receive a physiological signal through the internal tissue region or at a surface tissue region;
wherein the physiological signal comprises at least one physiological characteristic of the surface or internal tissue region; and
an antenna coupled to the array;
wherein the antenna is responsive to electromagnetic energy transmitted from the interrogator; and
wherein the electromagnetic energy powers the array with sufficient energy to power the emission or reception of the physiological signal through at least one of the sensor elements.

2. A system as recited in claim 1:
wherein the electromagnetic energy comprises RF energy;
wherein the sensor elements comprise a plurality of sensor or emitter electrodes; and
wherein the antenna comprises an RF coil configured to inductively power at least one of the electrodes.

3. A system as recited in claim 1:
wherein the electromagnetic energy comprises the sole source of power to the array.

4. A system as recited in claim 1:
wherein the electromagnetic waveform comprises a data signal; and
wherein the data signal comprises instructions readable by said processor for controlling the one or more elements.

5. A system as recited in claim 1:
wherein the electromagnetic energy comprises an optical waveform;
wherein the sensor elements comprise a plurality of optical sensors or emitters; and
wherein the antenna comprises an optical receiver configured to inductively power at least one of the optical sensors or emitters.

6. A system as recited in claim 1:
wherein the electromagnetic energy comprises an acoustic waveform;
wherein the sensor elements comprise a plurality of acoustic transducers; and
wherein the antenna comprises a transducer configured to inductively power at least one of the acoustic transducers.

7. A system as recited in claim 1, wherein said sensors elements are selected from the group of sensors consisting essentially of temperature sensors, moisture sensors, pressure sensors, bioelectric impedance sensors, electrical capacitance sensors, spectroscopic sensors, and optical sensors.

8. A system as recited in claim 4, wherein the array further comprises a signal demodulator to demodulate the electromagnetic signal for processing by the processor.

9. A system as recited in claim 8, wherein the array further comprises a signal modulator for transmitting a return data signal relating to said physiological characteristic from the array to the interrogator.

10. A system as recited in claim 1:
wherein the sensor elements are disposed at intersections of row and column transmission lines; and
wherein said transmission lines are coupled to said processor for individual control of the sensor elements.

11. A system as recited in claim 1:
wherein the array is configured to comprise at least one emitter element configured to emit a signal into the internal tissue region and at least on sensor element configured to receive a reflected signal from said tissue region; and
wherein the reflected signal comprises at least one physiological characteristic of said tissue region.

12. A system as recited in claim 1, wherein the sensor array comprises a first sensor array, the system further comprising:
a second array of sensor elements;
the second array configured to be positioned external to and adjacent the patient's skin;
the second array comprising:
a plurality of sensor elements; and
a processor connected to the plurality of sensor elements;
said processor configured to communicate with at least one of the sensors elements in the array;
wherein at least one sensor element of the second array is configured to emit a transmissive signal through the internal tissue region for reception by at least one sensor element in the first sensor array;
wherein physiological signal comprises at least one physiological characteristic of the internal tissue region.

13. A system as recited in claim 12, further comprising:
a second antenna coupled to the second array;
wherein the second antenna is responsive to electromagnetic energy transmitted from the interrogator; and
wherein the electromagnetic energy powers the second array with sufficient energy to power the emission of the transmitted signal through the internal tissue region to the first array.

14. A system as recited in claim 1, further comprising:
an implant disposed at or near the internal tissue region;
wherein the implant comprises at least one sensor element configured to emit a transmissive signal through the internal tissue region for reception by at least one sensor element in the second sensor array.

15. A system as recited in claim 14, further comprising:
a second antenna coupled to the implant;
wherein the second antenna is responsive to electromagnetic energy transmitted from the interrogator; and
wherein the electromagnetic energy powers the second antenna with sufficient energy to power the emission of the transmitted signal through the internal tissue region to the first array.

16. A method for acquiring one or more biological characteristics of a surface or internal tissue region of a patient, comprising:
positioning a sensor array external to and adjacent to a region of the patient's skin;
wherein the array comprises a plurality of sensor elements connected to a processor;
positioning an interrogator in proximity to said array;
the interrogator configured to transmit energy in the form of an electromagnetic waveform;
transmitting an electromagnetic signal from the interrogator;
receiving the electromagnetic signal via an antenna coupled to the array;
inductively powering the array via the electromagnetic signal; and
instructing the array via the electromagnetic signal to emit or receive a physiological signal through the internal tissue region or at a surface tissue region;
wherein the physiological signal comprises at least one physiological characteristic of the surface or internal tissue region.

17. A method as recited in claim 16:
wherein the electromagnetic energy comprises RF energy and the antenna comprises an RF coil;
wherein the array comprises a plurality of sensor or emitter electrodes; and
wherein inductively powering the array comprises powering the RF coil with sufficient energy to power at least one of the sensor or emitter electrodes.

18. A method as recited in claim 16:
wherein the electromagnetic energy comprises the sole source of power to the array.

19. A method as recited in claim 16:
wherein the electromagnetic signal comprises a data signal; and
wherein instructing the array comprises reading the data signal with said processor and operating at least one sensor element in the array based on one or more instructions is said data signal.

20. A method as recited in claim 16, wherein said sensor array comprises sensors are selected from the group of sensors consisting essentially of temperature sensors, moisture sensors, pressure sensors, bioelectric impedance sensors, electrical capacitance sensors, spectroscopic sensors, and optical sensors.

21. A method as recited in claim 19, further comprising:
demodulating the electromagnetic signal for processing by the processor.

22. A method as recited in claim 21, further comprising:
modulating a return signal relating to said physiological characteristic for transmission to the interrogator.

23. A method as recited in claim 16:
wherein the sensor elements are disposed at intersections of row and column transmission lines; and
wherein said transmission lines are coupled to said processor for individual control of the sensor elements.

24. A method as recited in claim 16, further comprising:
emitting a signal into the internal tissue region; and
receiving a reflected signal from said tissue region;
wherein the reflected signal comprises at least one physiological characteristic of said tissue region.

25. A method as recited in claim 16, wherein the sensor array comprises a first sensor array, the method further comprising:
positioning a sensor array external to and adjacent to a region of the patient's skin; and
emitting a transmissive physiological signal from the second sensor array through the internal tissue region for reception by the first sensor array;
wherein the physiological signal comprises at least one physiological characteristic of the internal tissue region.

26. A method as recited in claim 25:
wherein a second antenna is coupled to the second sensor array;
wherein the second antenna is responsive to electromagnetic energy transmitted from the interrogator; and
wherein the method further comprises powering the second sensor array with sufficient energy to power the emission of the transmitted physiological signal through the internal tissue region to the first array.

27. A method as recited in claim 16, further comprising:
delivering an implant at or near the internal tissue region; and
emitting a transmissive physiological signal from the implant through the internal tissue region for reception by the second sensor array.

28. A method as recited in claim 27, wherein the implant comprises a second antenna responsive to electromagnetic energy transmitted from the interrogator, the method further comprising;
powering the second antenna with sufficient energy to power the emission of the transmitted physiological signal through the internal tissue region to the first array.

29. A transdermal sensor system for acquiring one or more biological characteristics of an internal tissue region of a patient, comprising:
an interrogator configured to transmit energy in the form of an electromagnetic waveform;
an external sensor array;
an implant disposed at or near the internal tissue region;
wherein the implant comprises at least one internal sensor element configured to exchange a transmissive physiological signal through the internal tissue region with the external sensor array;
wherein the physiological signal comprises at least one physiological characteristic of the internal tissue region;
wherein the implant comprises an internal antenna responsive to electromagnetic energy transmitted from the interrogator; and
wherein the electromagnetic energy powers the implant with sufficient energy to power the exchange of the physiological signal through the at least one internal sensor element.

30. A system as recited in claim 29:
wherein said external sensor array comprises:
a substrate configured to be positioned external to and adjacent the patient's skin;
a plurality of external sensor elements coupled to the substrate; and
an array processor coupled to the substrate and connected to the plurality of external sensor elements;
said array processor configured to communicate with at least one of the external sensor elements in the array;
wherein the external sensor elements are configured to emit or receive the physiological signal;
an external antenna coupled to the array;
wherein the external antenna is responsive to electromagnetic energy transmitted from the interrogator; and
wherein the electromagnetic energy powers the array with sufficient energy to power the exchange of the transmissive physiological signal with the implant.

31. A system as recited in claim 30:
wherein the at least one internal sensor element comprises an emitter;
wherein at least one of the external sensor elements comprises a sensor; and
wherein the implant is configured to emit the transmissive physiological signal through the internal tissue region from the emitter for reception by the sensor of the external sensor array.

32. A system as recited in claim 30:
wherein the at least one internal sensor element comprises a sensor;
wherein at least one of the external sensor elements comprises an emitter; and
wherein the external sensor array is configured to emit the transmissive physiological signal through the internal tissue region from the emitter for reception by the sensor of the implant.

33. A system as recited in claim 30:
wherein the electromagnetic energy comprises RF energy;
wherein the external and internal sensor elements comprise sensor or emitter electrodes; and
wherein the external and internal antennas comprise RF coils configured to inductively power the sensor or emitter electrodes.

34. A system as recited in claim 30:
wherein the electromagnetic energy comprises the sole source of power to the array.

35. A system as recited in claim 30:
wherein the implant comprises an implant processor coupled to the at least one sensor element;
said implant processor configured to communicate with the at least one sensor element;
wherein the electromagnetic waveform comprises a data signal; and
wherein the data signal comprises instructions readable by said implant processor and said array processor for controlling at least one sensor element.

36. A system as recited in claim 30:
wherein the electromagnetic energy comprises an optical waveform;
wherein the sensor elements comprise a plurality of optical sensors or emitters; and
wherein the external and internal antennas comprise an optical receiver configured to inductively power at least one of the optical sensors or emitters.

37. A system as recited in claim 30:
wherein the electromagnetic energy comprises an acoustic waveform;
wherein the sensor elements comprise a plurality of acoustic transducers; and
wherein the external and internal antennas comprise a transducer configured to inductively power at least one of the acoustic transducers.

38. A system as recited in claim 29, wherein said sensors elements are selected from the group of sensors consisting essentially of temperature sensors, moisture sensors, pressure sensors, bioelectric impedance sensors, electrical capacitance sensors, spectroscopic sensors, and optical sensors.

39. A system as recited in claim 35, wherein the external array and implant each further comprise a signal demodulator to demodulate the electromagnetic signal.

40. A system as recited in claim 39, wherein the external array and implant each further comprise a signal modulator for transmitting a return data signal relating to said physiological characteristic from either the external array or the implant to the interrogator.

41. A system as recited in claim 29:
wherein the implant is disposed on an internally implanted prosthetic device;
wherein the internal sensor element is configured to exchange a transmissive physiological signal through at least a portion of the internally implanted prosthetic device with the external sensor array; and
wherein the a transmissive physiological signal relates to a physiological characteristic of the internally implanted prosthetic device.

42. A method for acquiring one or more biological characteristics of an internal tissue region of a patient, comprising:
positioning a sensor array external to and adjacent to a region of the patient's skin;
delivering an implant to a location at or near the internal tissue region;
positioning an interrogator in proximity to said array;
the interrogator configured to transmit energy in the form of an electromagnetic waveform;
wherein the implant comprises an internal antenna responsive to electromagnetic energy transmitted from the interrogator;
transmitting an electromagnetic signal from the interrogator;
receiving the electromagnetic signal via the internal antenna;
inductively powering the implant via the electromagnetic signal; and
instructing the implant via the electromagnetic signal to exchange a physiological signal with the external array through at least a portion of the internal tissue region;
wherein the physiological signal comprises at least one physiological characteristic of the internal tissue region.

43. A method as recited in claim 42, wherein the implant comprises at least one internal sensor element configured to exchange a transmissive physiological signal through the internal tissue region with the external sensor array;
wherein the implant comprises an internal antenna responsive to electromagnetic energy transmitted from the interrogator; and
wherein the electromagnetic energy powers the implant with sufficient energy to power the exchange of the physiological signal through the at least one internal sensor element.

44. A method as recited in claim 43:
wherein said external sensor array comprises a plurality of external sensor elements configured to emit or receive the physiological signal, an external antenna coupled to the array, and an array processor configured to communicate the antenna and at least one of the external sensor elements in the array;
wherein the external antenna is responsive to electromagnetic energy transmitted from the interrogator; and
wherein the electromagnetic energy powers the array with sufficient energy to power the exchange of the transmissive physiological signal with the implant.

45. A method as recited in claim 42:
wherein exchanging the physiological signal comprises emitting the transmissive physiological signal from the implant through the internal tissue region for reception by the external sensor array.

46. A method as recited in claim 42:
wherein exchanging the physiological signal comprises emitting the transmissive physiological signal from the external sensor array through the internal tissue region for reception by the implant.

47. A method as recited in claim 44:
wherein the electromagnetic energy comprises RF energy;
wherein the external and internal sensor elements comprise sensor or emitter electrodes; and
wherein inductively powering the implant comprises powering the external and internal antennas to inductively power the sensor or emitter electrodes.

48. A method as recited in claim 44:
wherein the electromagnetic signal comprises a data signal and the implant comprises an implant processor coupled to the at least one internal sensor element; and
wherein instructing the implant comprises reading the data signal with said implant processor and operating the at least one sensor element based on one or more instructions in said data signal.

49. A method as recited in claim 42, wherein said implant and external sensor array are selected from a group of sensors consisting essentially of temperature sensors, moisture sensors, pressure sensors, bioelectric impedance sensors, electrical capacitance sensors, spectroscopic sensors, and optical sensors.

50. A method as recited in claim 48, further comprising:
demodulating the electromagnetic signal for processing by the implant processor.

51. A method as recited in claim 48, further comprising:
modulating a return signal relating to said physiological characteristic for transmission from the implant to the interrogator.

52. A method as recited in claim 48, further comprising:
modulating a return signal relating to said physiological characteristic for transmission from the external sensor array to the interrogator.

53. A method as recited in claim 42, further comprising:
delivering a second implant at or near the internal tissue region;
exchanging a second transmissive physiological signal through the internal tissue region with the external sensor array.

54. An interrogatable sensor system for acquiring one or more biological characteristics of an internal tissue region of a patient, comprising:
an interrogator configured to be positioned at a location external to the body of the patient and transmit energy in the form of an electromagnetic waveform;
a first implant configured to be disposed at or near the internal tissue region;
wherein the first implant comprises a sensor element configured to receive a physiological signal through at least a portion of the internal tissue region;
wherein the physiological signal emanating within the body of the patient and comprising at least one physiological characteristic of the internal tissue region;
wherein the first implant comprises an antenna responsive to electromagnetic energy transmitted from the interrogator; and
wherein the electromagnetic energy powers the implant with sufficient energy to power the receipt of the physiological signal through the sensor element.

55. A system as recited in claim 54:
wherein the first implant further comprises an emitter element coupled to the antenna; and
wherein the emitter element is configured to emit a physiological signal into at least a portion of the internal tissue region; and
wherein the physiological signal comprises at least one physiological characteristic of the internal tissue region.

56. A system as recited in claim 55:
wherein the sensor element is configured to receive a reflected signal from the internal tissue region; and
wherein the reflected signal emanates from the emitter.

57. A system as recited in claim 55:
wherein the electromagnetic energy comprises RF energy;
wherein the sensor element and emitter element comprise sensor or emitter electrodes; and
wherein the antenna comprises an RF coil configured to inductively power at least one of the electrodes.

58. A system as recited in claim 54:
wherein the electromagnetic energy comprises the sole source of power to the array.

59. A system as recited in claim 54:
wherein the first implant further comprises a first processor coupled to the internal antenna and sensor element;
wherein the electromagnetic waveform comprises a data signal; and
wherein the data signal comprises instructions readable by said first processor for controlling the sensor elements.

60. A system as recited in claim 55:
wherein the electromagnetic energy comprises an optical waveform;
wherein the sensor element and emitter element comprise optical sensors or emitters; and
wherein the internal antenna comprises an optical receiver configured to inductively power at least one of the optical sensor or emitter.

61. A system as recited in claim 55:
wherein the electromagnetic energy comprises an acoustic waveform;
wherein the sensor element and emitter element comprise an acoustic transducer; and
wherein the internal antenna comprises a transducer configured to inductively power at least one of the acoustic transducers.

62. A system as recited in claim 54, wherein said sensor element is selected from the group of sensors consisting essentially of temperature sensors, moisture sensors, pressure sensors, bioelectric impedance sensors, electrical capacitance sensors, spectroscopic sensors, and optical sensors.

63. A system as recited in claim 59, wherein the first implant further comprises a signal demodulator to demodulate the electromagnetic signal for processing by the first processor.

64. A system as recited in claim 59, wherein the first implant further comprises a signal modulator for transmitting a return data signal relating to said physiological characteristic from the array to the interrogator.

65. A system as recited in claim 59, further comprising:
a second implant configured to be disposed at or near the internal tissue region;
wherein the second implant comprises an emitter element configured to emit a physiological signal through at least a portion of the internal tissue region;
wherein the physiological signal comprises at least one physiological characteristic of the internal tissue region;
wherein the second implant comprises an antenna responsive to electromagnetic energy transmitted from the interrogator; and
wherein the electromagnetic energy powers the second implant with sufficient energy to power the transmission of the physiological signal through at least a portion of the internal tissue region to be received by the first implant.

66. A system as recited in claim 54, wherein the first implant further comprises:
a stent structure configured to be delivered to a location within the body of the patient;
the stent structure comprising a central channel configured to allow fluid communication therethrough;
wherein the sensor element comprises a first sensor element configured to receive a first physiological signal relating to the fluid communication through the stent;
the stent structure configured to house the first sensor element and a second sensor element;
the sensor configured to receive a second physiological signal relating to the fluid communication through the stent.

67. A system as recited in claim 66:
wherein the stent further comprises a heating element disposed between the first sensor element and the second sensor element;
wherein first sensor element is configured to receive a first temperature measurement and the second sensor element is configured to receive a second temperature measurement; and
wherein the first and second measurements relate to a flowrate of the fluid communication through the stent.

68. A method for acquiring one or more biological characteristics of an internal tissue region of a patient, comprising:
positioning an interrogator at a location external to the body of the patient;
the interrogator configured to transmit energy in the form of an electromagnetic waveform;
delivering a first implant to a location at or near the internal tissue region;
wherein the first implant comprises a sensor element configured to receive a physiological signal through at least a portion of the internal tissue region;
wherein the first implant comprises an antenna responsive to electromagnetic energy transmitted from the interrogator;
transmitting an electromagnetic signal from the interrogator;
receiving the electromagnetic signal via the antenna;
inductively powering the first implant via the electromagnetic signal; and
instructing the implant via the electromagnetic receive a physiological signal emanating within the body of the patient and comprising at least one physiological characteristic of the internal tissue region;
wherein the electromagnetic energy powers the implant with sufficient energy to power the receipt of the physiological signal through the sensor element.

69. A method as recited in claim 68, wherein the first implant further comprises an emitter element coupled to the antenna, the method further comprising:
instructing the first implant via the electromagnetic signal to emit a physiological signal into the body of the patient from the emitter element;
wherein the electromagnetic energy powers the implant with sufficient energy to power the transmission of the physiological signal.

70. A method as recited in claim 69;
wherein the sensor element is configured to receive a reflected signal from the internal tissue region; and
wherein the reflected signal emanates from the emitter.

71. A method as recited in claim 69:
wherein the electromagnetic energy comprises RF energy;
wherein the sensor element and emitter element comprise sensor or emitter electrodes; and
wherein inductively powering the implant comprises powering the antenna to inductively power at least one of the electrodes.

72. A method as recited in claim 68:
wherein the electromagnetic energy comprises the sole source of power to the array.

73. A method as recited in claim 68:
wherein the first implant further comprises a first processor coupled to the antenna and sensor element;
wherein the electromagnetic waveform comprises a data signal; and
wherein instructing the implant comprises reading the data signal with said first processor and operating the sensor element based on one or more instructions in said data signal.

74. A method as recited in claim 68, wherein said sensor is selected from a group of sensors consisting essentially of temperature sensors, moisture sensors, pressure sensors, bioelectric impedance sensors, electrical capacitance sensors, spectroscopic sensors, and optical sensors.

75. A method as recited in claim 73, further comprising:
demodulating the electromagnetic signal for processing by the first processor.

76. A method as recited in claim 73, further comprising:
modulating a return signal relating to said physiological characteristic for transmission from the implant to the interrogator.

77. A method as recited in claim 68, further comprising:
delivering a second implant at or near the internal tissue region;
wherein the second implant comprises an emitter element configured to emit a physiological signal through at least a portion of the internal tissue region;
wherein the physiological signal comprises at least one physiological characteristic of the internal tissue region;
wherein the second implant comprises an antenna responsive to electromagnetic energy transmitted from the interrogator; and
powering the second implant via the electromagnetic energy sufficiently to power the transmission of the physiological signal through at least a portion of the internal tissue region to be received by the first implant.
